SEQUENCE LISTING (13 sequences)

SEQ ID NO: 1 - DNA, 20 bp, Artificial Sequence
  Primer sequence
gcacacacgtaactgaccac

SEQ ID NO: 2 - DNA, 20 bp, Artificial Sequence
  Primer Sequence
cattgggtacgtagcccacc

SEQ ID NO: 3 - DNA, 20 bp, Artificial Sequence
  Primer Sequence
tgactttgtcacagcccaag

SEQ ID NO: 4 - DNA, 20 bp, Artificial Sequence
  Primer Sequence
tctctgctccccacctctaa

SEQ ID NO: 5 - DNA, 23 bp, Artificial Sequence
  Primer Sequence
gctaactagggaacccactgctt

SEQ ID NO: 6 - DNA, 22 bp, Artificial Sequence
  Primer Sequence
caacagacgggcacacactact

SEQ ID NO: 7 - DNA, 30 bp, Artificial Sequence
  Probe Sequence provided sequence terminates with 5′-[FAM] and 3′-[TAM] labels (not shown)
agcctcaataaagcttgccttgagtgcttc

SEQ ID NO: 8 - DNA, 24 bp, Artificial Sequence
  Primer Sequence
caacagccacaacgtctatatcat

SEQ ID NO: 9 - DNA, 21 bp, Artificial Sequence
  Primer Sequence
atgttgtggcggatcttgaag

SEQ ID NO: 10 - DNA, 25 bp, Artificial Sequence
  Probe Sequence. Provided sequence terminates with 5′-[FAM] and 3′-[TAMRA] labels (not shown)
ccgacaagcagaagaacggcatcaa

SEQ ID NO: 11 - DNA, 22 bp, Artificial Sequence
  Primer Sequence
gactcatgaccacagtccatgc

SEQ ID NO: 12 - DNA, 21 bp, Artificial Sequence
  Primer Sequence
agaggcagggatgatgttctg

SEQ ID NO: 13 - DNA, 25 bp, Artificial Sequence
  Probe Sequence provided sequence terminates with 5′-[JOE] and 3′-[TAM] labels (not shown)
catcactgccacccagaagactgtg

(19) United States
(12) Patent Application Publication
(10) Pub. No.: US 2013/0261137 A1
(43) Pub. Date: Oct. 3, 2013

(54) Methods, Compounds and Compositions Relating to Activating a Latent Virus

(71) Applicants: Edward Hiu Fung Tsao, London (GB); Paul Kellam, London (GB); Richard Gareth Jenner, London (GB)

(72) Inventors: Edward Hiu Fung Tsao, London (GB); Paul Kellam, London (GB); Richard Gareth Jenner, London (GB)

(73) Assignee: ULC BUSINESS PLC, London (GB)

(21) Appl. No.: 13/823,344

(22) PCT Filed: Sep. 9, 2011
(86) PCT No.: PCT/GB11/51693

§ 371 Date: Jun. 17, 2013

(30) Foreign Application Priority Data
Sep. 14, 2010 (GB) 1015305.4

(51) Int. Cl. A61K 31/407; A61K 31/522; A61K 31/353; A61K 45/06

(52) U.S. Cl. CPC A61K 31/407; A61K 45/06; A61K 31/522; A61K 31/353
USPC 514/263.38; 514/411

(57) ABSTRACT

The present invention relates to, inter alia, an anti-microtubule agent for use in a method of treating a subject having a latent virus, the method comprising: administering the anti-microtubule agent and an anti-viral agent to the subject.

### TECHNICAL FIELD

The present invention relates to activating a latent virus and, in certain embodiments, treating a latent virus.

### BACKGROUND

Viruses that establish lifelong persistence in a host often utilize latency, typified by the ability of the viral genomes to remain dormant in infected cells, to escape immune system recognition and elimination. Operationally this is achieved by the expression of a restricted subset, or sometimes a complete absence, of viral proteins and RNA. Therefore, no virions are typically produced in latently infected cells. Crucially, latent viral genomes possess the capacity to reactivate resulting in the expression of the full repertoire of viral genes, leading ultimately to the production of new progeny viruses. Latency is a key characteristic of infection by herpes viruses and by human immunodeficiency virus type 1 (HIV-1). Kaposi's sarcoma-associated herpes virus (KSHV) is the leading cause of cancer in immunodeficient individuals and the tumors predominantly consist of latently infected cells (see review¹, which is incorporated herein by reference in its entirety). HIV-1 can remain latent in quiescent CD4+ T-cells. This latent viral reservoir is stable, with a half-life of 44 months, guaranteeing lifelong persistence (see references^{2,3}, which are each incorporated herein by reference in their entirety).

Latency poses a problem for virus eradication from the host as both immune responses and all currently licensed antiviral drugs target viral proteins expressed only during their lytic cycle. For example, antiherpetic drug such as ganciclovir (GCV) is effective in preventing human cytomegalovirus (HCMV) disease and is licensed for use in solid organ transplant patients but it does not clear latent infection. Following initial anti-HCMV therapy, the risk of developing recurrent HCMV disease is higher in patients receiving placebo compared with patients receiving continuous oral GCV (see reference⁴, which is incorporated herein by reference in its entirety). Similarly, highly-active anti-retroviral therapy (HAART) is generally successful in reducing plasma HIV to undetectable levels but has no effect on latent proviral DNA. The reactivation of latently infected cells causes plasma virus load to rebound within two weeks of HAART ending (see reference⁵, which is incorporated herein by reference in its entirety). There is a need therefore to develop therapeutics that switch latent viruses into a replicative cycle, thus rendering the infected cells susceptible to the immune system or therapy (see references⁶⁻⁸ and review⁵, which are incorporated herein by reference in their entirety). This so-called adjunctive antiviral therapy offers a way to purge viral reservoirs, whilst preventing the full maturation of infectious virions with existing antiviral drugs thus blocking new cell infection.

Viruses rely on the cellular machinery of the host to complete their life cycle thereby allowing the therapeutic targeting of host proteins and functions that support virus replication (see reference⁹, which is incorporated herein by reference in its entirety). The modulation of such proteins can attenuate virus replication with a reduced likelihood of selecting for virus resistance. Targeting host proteins utilized by a wide range of viruses may lead to broad spectrum antiviral agents. Knowledge of cellular factors or proteins required for virus replication has led to the repositioning of existing drugs as antivirals often with rapid in vivo testing (see reference¹⁰, which is incorporated herein by reference in its entirety). Recently, RNAi screens have revealed the enormous potential for therapeutically targeting host proteins for antiviral effect¹¹⁻¹³. Similarly, host cell proteins could be targeted to reactivate latent viruses. Indeed, it has been shown that broad spectrum but toxic compounds such as histone deacetylase (HDAC) inhibitors reactivate HIV-1 (see references^{6,14}, which are incorporated herein by reference in their entirety) and the oncogenic human herpes viruses Epstein Barr virus (EBV) and KSHV (see references^{7,8}, which are incorporated herein by reference in their entirety). Importantly, induction of the viral lytic cycle in EBV- or KSHV-associated tumors should result in enhanced susceptibility to GCV that preferentially kill cells containing reactivating herpesviruses.

It is an aim of the present invention to provide an alternative or improved agent for reactivating a latent virus.

Choudhary et al have published a paper entitled Curing HIV: Pharmacologic Approaches to Target HIV-1 Latency (Annu. Rev. Pharmacol. Toxicol. 2011. 51:397-418).

Sigal et al have published a paper entitled Cell-to-cell spread of HIV permits ongoing replication despite antiretroviral therapy (2011 Aug. 17; 477(7362):95-8).

### SUMMARY OF THE INVENTION

In a first aspect, the present invention provides a method of activating a latent virus in a subject comprising
administering an anti-microtubule agent to the subject.

In a second aspect, the present invention provides a method of treating a subject having a latent virus comprising
administering an anti-microtubule agent and an anti-viral agent to the subject.

In a third aspect, the present invention provides an anti-microtubule agent for use in a method of treating a subject having a latent virus, the method comprising:
administering the anti-microtubule agent and an anti-viral agent to the subject.

In a fourth aspect, the present invention provides a composition comprising an anti-microtubule agent and an anti-viral agent.

In a fifth aspect, the present invention provides a kit, the kit comprising:
an anti-microtubule agent,
an anti-viral agent, and
instructions on how to treat the virus using the anti-microtubule agent, and the anti-viral agent.

In an embodiment, the latent virus is selected from a retrovirus and a herpes virus. The retrovirus may be selected from the human immunodeficiency virus type I (HIV-1), human immunodeficiency virus type II (HIV-2), and Human T-lymphotropic virus Type I and II (HTLV-1, -2). The retrovirus may be a HIV-1 of Group M, and optionally of a subtype selected from A, B, C and A/G. The herpes virus may be selected from herpes simplex virus-1, herpes simplex virus-2, varicella zoster virus, Epstein-Barr virus, cytomegalovirus, roseolovirus, Kaposi's sarcoma-associated herpesvirus, and all animal herpesviruses including, but not limited to, Equine herpesvirus, Marek's Disease virus, Porcine herpesvirus and Porcine cytomegalovirus.

In an embodiment, the anti-microtubule agent is a compound of the formula (I)

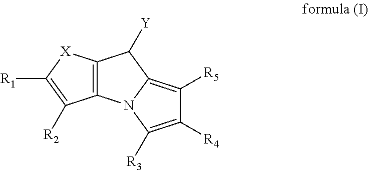

wherein X is selected from S, O, N—R₆, wherein R₆ is selected from H or optionally substituted alkyl,
—Y is selected from ═O, —OH, ═N—R₇, wherein R₇ is selected from H or optionally substituted alkyl,

R₂ is optionally substituted aryl or heteroaryl,

R₁, R₃, R₄ and R₅ are each independently selected from hydrogen, halogen, optionally substituted alkyl, optionally substituted alkoxy, optionally substituted amino group, a nitro group, optionally substituted aryl and optionally substituted heteroaryl;
or pharmaceutically acceptable salts thereof, or prodrugs thereof.

### BRIEF DESCRIPTION OF THE FIGURES

FIGS. 1(A) to 1(C) relate to a primary screen identifying 70 hits that induce expression of a KSHV lytic marker. FIG. 1(A) shows a schematic diagram of the rKSHV.219 virus. The lytic reporter cassette is inserted between ORF 57 and ORF K9 of the KSHV genome¹⁶. Representative results of the KSHV reactivation screen is depicted in images (B) and bar-chart (C). DMSO and TPA are the negative and positive controls, respectively. Compound 01_G10 has no effect, compound 01_E6 induces 0.7% RFP expression and is scored “moderate” and compound 01_C2 induces 3.07% RFP expression and is scored “high” (y-axis, % RFP-positive cells; x-axis, compound co-ordinate).

FIG. 2 shows the KSHV induction kinetics by active compounds. Representative western blot showing the absence of KSHV RTA (110 kDa) induction by DMSO. RTA induction by TPA and SB displayed immediate- and delayed-kinetics, respectively. Results of three primary hits, denoted in the format PH (number), are shown.

FIGS. 3(A) to 3(D) show that the compound UCLB-15026 induces the KSHV lytic cycle resulting in increased susceptibility to GCV-mediated cytotoxicity. Structures and NSC numbers of compound PH30 and compound UCLB-15026 obtained from the PubChem project are shown in FIGS. 3(A) and 3(B), respectively. FIG. 3(C) shows the semi-quantitative RT-PCR analysis of KSHV ORF 29 using serially-diluted cDNA obtained from JSC-1 treated with DMSO, TPA (20 ng/ml), UCLB-15026 or Taxol at different concentration. L denotes the 100 bp DNA ladder and the numbers denote size of the PCR products (in bp). Input cDNA was normalized to a housekeeping gene (β₂M). Primers for both genes span across intron(s) and only amplify cDNA under the PCR conditions used. FIG. 3(D) shows cell viability as measured by CellTiterGlo at 48 and 72 h of JSC-1 cells pre-treated with either UCLB-15026 (800 nM) or taxol (Tx, 80 nM) for 24 h, and then replaced with media containing no GCV (white), GCV at 20 μM (grey) or at 100 μM (black). The y-axis represents % concentration of ATP (proportional to the number of live cells) and is normalized to the no GCV (white) control. **p-value<0.01 (two-tailed, paired).

FIG. 4 shows that compound UCLB-15026 induces HIV-1 LTR activity. J-Lat A72 cells treated with UCLB-15026 at different concentrations and measured at either 24 (white) or 40 h (black). The y-axis represents % GFP-positive cells as normalized to the TPA control.

FIG. 5 shows that anti-microtubule agents UCLB-15026 and taxol both induce RTA expression in PEL irrespective of EBV co-infection. FIG. 5(A) shows confocal images showing distinct microtubule network (green staining, or light grey staining if printed in black and white, —tubulin) in JSC-1 cells treated with DMSO, and dispersed tubulin staining in cells treated with UCLB-15026 indicating the disruption of microtubule network. DAPI staining (which appeared blue) showed nucleus of PEL cells. FIG. 5(B) shows that UCLB-15026 and taxol (Tx) induce RTA expression in JSC-1 in the nanomolar range. This is measured using JSC-1 cells stably transduced with a KSHV lytic PAN-YFP reporter. The y-axis represents % YFP-positive cells as normalized to the TPA control. FIG. 5(C) shows that both UCLB-15026 (800 nM) and taxol (Tx; 80 nM) activate RTA expression in JSC-1 and BCBL-1 (both stably transduced with a KSHV lytic PAN-LUC reporter). The y-axis represents % RLU (relative luciferase activity) as normalized to the TPA control/10⁵ cells.

FIG. 6 shows the cytotoxic properties of ganciclovir (GCV), UCLB-15026, and taxol (Tx). JSC-1 cells treated with DMSO for 24 h, and cultured in fresh media containing no drug (circles and dashed line), or GCV at 20 μM (triangles and dashed line) or 100 μM (squares and dashed line), as well as JSC-1 cells treated with UCLB-15026 (800 nM; circles and solid line) or Tx (80 nM; triangles and solid line) for 24 h and cultured in fresh drug free media. All samples were measured for viability at 24, 48, and 72 h. The y-axis represents the concentration of ATP (proportional to number of live cells) in log₁₀ scale.

FIG. 7 shows HIV-1 LTR induction by genistein (GN; 100 μM). HIV-1 LTR activities in A72 cells as measured at 24 h post-treatment. The y-axis represents % GFP-positive cells as normalized to the TPA control.

FIG. 8 shows KSHV lytic cycle induction by genistein. Effect of geistein (100-μM) on levels of YFP expression in JSC-1 PYFP cells. The y-axis represents % YFP-positive cells as normalized to the TPA control.

FIG. 9 shows KSHV RTA induction by UCLB-15026 (0.8 μM) requires ERK activity. FIG. 9(A) RTA induction by UCLB-15026 with or without JNK inhibitor SP600125 (JNKi, 20 μM), p38 inhibitor SB203580 (p38i, 20 μM), or ERK inhibitor U0126 (ERKi, 10 μM). FIG. 9(B) Dose-effect relationship of ERKi on KSHV RTA induction by UCLB-15026. FIG. 9(C) Effect of another ERK-specific inhibitor PD0325901 (ERK PD, 0.1 μM) on KSHV reactivation by UCLB-15026. FIG. 9(D) Effect of mock (M), ectopic expression of a constitutively active (ED), or a dominant negative (AA) MEK mutants on RTA induction by UCLB-15026. All data are shown as relative RTA activation compared with DMSO control and plotted as mean±SD from three independent experiments, except for FIG. 3D where data are plotted as mean±SD from three replicates per treatment and are representative of two independent experiments.

FIG. 10. shows UCLB-15026 induces HIV-1 LTR through the ERK pathway and potentiate activities of other small molecule activators. FIG. 10(A) Effect of UCLB-15026 (4 μM) on HIV-1 LTR activity in A72 (as measured by GFP expression), ACH2, or U1 cells (as measured by TAR RNA copy number) and its modulation by the ERK inhibitor U0126 (ERKi, 10 μM). FIG. 10(B) Effect of UCLB-15026, in the absence or presence of ERKi, on LGIT cells containing HIV-1 LTR of either subtype A, C or A/G. FIG. 10(C) Effect of prostratin (PS, 1 μM), UCLB-15026, valproic acid (VPA, 1.5 mM) used either singly or in combination on HIV-1 LTR activity as measured by qPCR for TAR RNA (ACH2 cells) or GFP (A72 cells). All data are shown as relative LTR activation compared with DMSO control and plotted as mean±SD from at least two independent experiments. FIG. 10(D) Effect of UCLB-15026 on HIV-1 LTR activity in PBMCs from HIV-1 positive individuals with undetectable viral load as measured by qPCR for TAR RNA (diamond—volunteer 1, triangle—volunteer 2, and square—volunteer 3). Each data point depicts the average of duplicate qPCR measurements, and the dotted line indicates the baseline of this qPCR assay. “15025” where it appears in this Figure should read “15026”.

FIG. 11 shows full induction of both the KSHV and HIV-1 promoters that control virus replication by UCLB-15026 requires MSK1. FIG. 11(A) Effect of UCLB-15026 (0.8 μM) on KSHV RTA and HIV-1 LTR activity and its modulation by the MSK1 inhibitor H89 (10 μM, black bar) and PKA inhibitor Rp-cAMP (100 μM, grey bar) compared with DMSO control (white bar). FIG. 11(B) Effect of ectopic expression of a wild-type (WT, white bar), or a dominant negative (DN, black bar) MSK1 mutants on KSHV RTA and HIV-1 LTR activity by UCLB-15026. All data are shown as relative induction of KSHV RTA or HIV-1 LTR compared with DMSO or WT controls and plotted as mean±SD from at least three independent experiments. ** denotes p-value<0.02 (two-tailed, paired T-test).

FIG. 12 shows KSHV reactivation by UCLB-15026 or taxol is independent of caspase activity. FIG. 12(A) Proportions of apoptotic JSC-1 cells in the absence (white), or presence (black) of pan-caspase inhibitor Q-VD-OPH (100 nM) as measured by flow cytometry at 40 h after treatment with UCLB-15026 (UCLB, 800 nM) or taxol (Tx, 80 nM). Data are shown as the percentage of live cells gated on forward and side scatter and plotted as mean±SEM. FIG. 12(B) The effect of pan-caspase inhibitor Q-VD-OPH (QVD) on RTA induction by UCLB-15026 or taxol at 40 hours post-treatment. Data are shown as relative RTA activation compared with the DMSO control and plotted as mean±SEM.

FIG. 13 shows U0126 (10 μM) treatment has no effect on the viability of JSC-1 PYFP cells. Cell viability was measured by trypan blue staining at 40 hours post-treatment and plotted as mean±SEM.

FIG. 14 shows other antimicrotubule compounds induce transcriptional activities of both the KSHV and HIV-1 promoters that control virus replication through the ERK pathway. FIG. 14(A) Effect of ERK inhibitor (ERKi) U0126 on HIV-1 LTR induction by genistein (GS, 100 μM), taxol (Tx, 0.8 μM) and vinblastine (VB, 0.8 μM). FIG. 14(B) Effect of ERKi on KSHV RTA induction by genistein (GS, 50 μM) and vinblastine (VB, 0.8 μM). All data are shown as relative LTR/RTA activation compared with DMSO control and plotted as mean±SEM from three independent experiments.

### DETAILED DESCRIPTION OF THE INVENTION

In a first aspect, the present invention provides a method of activating a latent virus in a subject comprising
administering an anti-microtubule agent to the subject.

In a second aspect, the present invention provides a method of treating a subject having a latent virus comprising
administering an anti-microtubule agent and an anti-viral agent to the subject.

In a third aspect, the present invention provides an anti-microtubule agent for use in a method of treating a subject having a latent virus, the method comprising:
administering the anti-microtubule agent and an anti-viral agent to the subject.

In a fourth aspect, the present invention provides a composition comprising an anti-microtubule agent and an anti-viral agent.

In a fifth aspect, the present invention provides a kit, the kit comprising:
an anti-microtubule agent,
an anti-viral agent, and
instructions on how to treat the virus using the anti-microtubule agent, and the anti-viral agent.

In a sixth aspect, the present invention provides a method of treating a subject having a latent virus comprising administering an agent that activates MSK-1 and an anti-viral agent to the subject. The agent that activates MSK-1 may or may not be an anti-microtubule agent. The agent that activates MSK-1 may be a compound of formula (I) as described herein. The latent virus may be as described herein.

The present inventors have found that anti-microtubule agents can activate a latent virus. Activating a latent virus indicates that the virus is induced into its replicative cycle. Once the latent virus has been activated, an anti-viral agent can act upon the virus. An anti-viral agent includes, but is not limited to an agent that can treat a virus when it is in its active state, for example when the virus is transcriptionally active in at least one or more cells in a subject.

In an embodiment, the latent virus is selected from a retrovirus and a herpes virus. The retrovirus may be selected from the human immunodeficiency virus type I (HIV-1), human immunodeficiency virus type II (HIV-2), and Human T-lymphotropic virus Type I and II (HTLV-1, -2). The herpes virus may be selected from herpes simplex virus-1, herpes simplex virus-2, varicella zoster virus, Epstein-Barr virus, cytomegalovirus, roseolovirus, Kaposi's sarcoma-associated herpesvirus, and all animal herpesviruses including, but not limited to, Equine herpesvirus, Marek's Disease virus, Porcine herpesvirus and Porcine cytomegalovirus.

Anti-microtubule agents are known in the art. Microtubules are components of the cytoskeleton in a cell. Microtubules are dynamic structures that undergo continual assembly and disassembly within the cell. They function both to determine cell shape and in a variety of cell movements, including some forms of cell locomotion, the intracellular transport of organelles, and the separation of chromosomes during mitosis.

Microtubules are formed from a globular protein called tubulin, which is a dimer formed from two polypeptides, α-tubulin and β-tubulin. The tubulin dimers polymerize end to end in protofilaments. The protofilaments then bundle into hollow cylindrical filaments. Typically, the protofilaments arrange themselves in an imperfect helix with one turn of the helix containing 13 tubulin dimers, each from a different protofilament. To perform their functions within a cell, the microtubules should be able to form, from the polymerisation of tubulin, and depolymerise as required. Anti-microtubule agents act either to depolymerise the microtubule structure or stabilise the microtubule structure to prevent its depolymerisation in the cell, either of which prevents the microtubules carrying out their normal function, e.g. in mitosis. Cancerous cells typically have a higher rate of mitosis compared to non-cancerous cells, so anti-microtubulin agents, which act to prevent or slow the mitosis, have found use in the treatment of cancer. Examples of anti-microtubule agents that depolymerise microtubules include Nocodazole and Colchicine. An example of an anti-microtubule agent that stabilises microtubules is taxol. An anti-microtubule agent may be a species that binds to tubulin.

The anti-microtubule agent may be (i) an agent that, in vivo or in vitro, prevents or reduces the rate of polymerisation of tubulin, compared to conditions in which the agent is absent, but are otherwise the same and under which tubulin will polymerise, (such agents can prevent or slow the formation of microtubulin and/or promote the depolymerisation of microtubulin), or (ii) an agent that, in vivo or in vitro, has a microtubule-stabilizing effect, such an agent may increase the rate of polymerisation of tubulin compared to conditions in which the agent is absent, but are otherwise the same and under which tubulin will polymerise. Conditions under (i) or (ii) above may, for example, be conditions in which a test is carried out using a receptacle that contains a mixture comprising 2 mg/ml tubulin (e.g. derived from the Porcine brain) at pH 6.9, 2.0 mM MgCl₂, 0.5 mM EGTA (ethylene glycol-bis(b-amino-ethyl ether) N,N,N′,N′-tetra-acetic acid), 1.0 mM GTP and 10 wt % glycerol, and in which, at the start of the test, the temperature of the mixture is 4° C., at which point the agent to be tested may be added and the temperature raised to 37° C. and then the polymerisation of tubulin measured over a desired period at 37° C., e.g. for 500 seconds or more, optionally 1000 seconds or more, optionally 1500 seconds or more. The rate of polymerisation of tubulin may be measured using any suitable method, for example suitable fluorescence techniques known to the skilled person, which may involve a suitable fluorescent reporter being present in the receptacle mentioned above. Assays for the identification of anti-microtubule agents are known to the skilled person. For example, the assay may be an assay for identifying if an agent prevents or reduces the rate of the polymerisation of tubulin compared to conditions in which the agent is absent (but which are otherwise the same and under which tubulin does polymerise). The assay may be an assay for identifying if an agent increases the rate of the polymerisation of tubulin compared to conditions in which the agent is absent (but which are otherwise the same and under which tubulin does polymerise). Suitable assays are described, for example in JBC. 260 (5): 2815-2895 (published in 1985, authored by Bonne et al. and entitled 4′,6-diamino-2-phenylindole, a fluorescent probe for tubulin and microtubules), which is incorporated herein in its entirety, or in accordance with the methods described in Mol Cancer Ther 2008; 7(1). January 2008 (authored by Huber et al and entitled 2[(1-Methylpropyl)dithio]-1H-imidazole inhibits tubulin polymerization through cysteine oxidation), which is incorporated herein by reference in its entirety; see, for example, the section in Huber et al with the heading Tubulin Polymerization Assays on page 145]. Suitable kits for use in such assays are commercially available, for example the Tubulin Polymerization Assay Kit from Cytoskeleton, Inc. (Porcine tubulin and fluorescence based), Cat. #BK011P; a full protocol for such an assay can be found at http://www.funakoshi.co.jp/data/datasheet/CYO/BK011P.pdf, which is incorporated by reference herein in its entirety. The assay may be an assay for identifying if an agent promotes depolymerisation of microtubule, an example tubulin depolymerisation assay is described on page 145 Mol Cancer Ther 2008; 7(1). January 2008, mentioned above. The type of tubulin used in the assay(s) may be any suitable type. An example is tubulin from the Porcine brain. Assays using tubulin from the Porcine brain can be used as an indication of whether an agent will act as an anti-microtubule agent in a human.

The anti-microtubule agent may be an agent selected from a compound of formula (I) below, genistein, vinca alkaloids (including, but not limited to, vincristine, vinblastine, vindesine and vinorelbin), taxotere, maytansin, rhizoxin, taxane compounds, and combinations thereof.

Optionally, the present invention excludes genistein for the activation of a latent HIV-1 virus.

Taxane compounds are known in the art and include, for example, paclitaxel (available as TAXOL® from Bristol-Myers Squibb, Princeton, N.J.), docetaxel (available as TAXOTERE® from Sanofi-aventis, Bridgewater, N.J.), and the like. Other taxane compounds that become approved by the U.S. Food and Drug Administration (FDA) or foreign counterparts thereof are may be used in the present invention. Other taxane compounds that can be used in the present invention include those described, for example, in 10th NCI-EORTC Symposium on New Drugs in Cancer Therapy, Amsterdam, page 100, Nos. 382 and 383 (Jun. 16-19, 1998).

Preferably, the anti-microtubule agent is an agent that prevents the polymerisation of tubulin, and/or promotes depolymerisation of tubulin.

In an embodiment, the anti-microtubule agent is a compound of the formula (I)

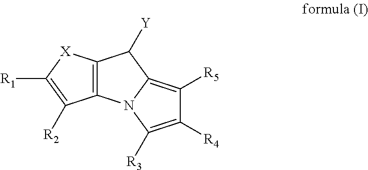

wherein X is selected from S, O, N—R₆, wherein R₆ is selected from H or optionally substituted alkyl,
—Y is selected from ═O, —OH, ═N—R₇, wherein R₇ is selected from H or optionally substituted alkyl,
R₂ is optionally substituted aryl or heteroaryl,
R₁, R₃, R₄ and R₅ are each independently selected from hydrogen, halogen, optionally substituted alkyl, optionally substituted alkoxy, optionally substituted amino group, a nitro group, optionally substituted aryl and optionally substituted heteroaryl;
or pharmaceutically acceptable salts thereof, or prodrugs thereof.

Preferably, X is S.

Preferably, —Y is =0.

Preferably, R₁, R₃, R₄ and R₅ are all H.

Preferably, R₂ is substituted aryl or heteroaryl. In an embodiment, X is S, —Y is ═O, R₁, R₃, R₄ and R₅ are all H, and R₂ is optionally substituted phenyl, more preferably substituted phenyl.

Preferably R₂ is a group of the formula (II)

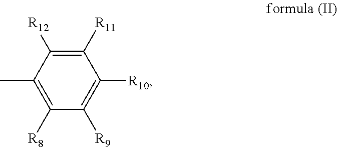

wherein R₈, R₉, R₁₀, R₁₁, R₁₂ are each independently selected from hydrogen, halogen, optionally substituted alkyl, nitro, cyano, hydroxy, optionally substituted alkoxy, optionally substituted amino, carboxy, alkoxycarbonyl, methylenedioxy, ethylenedioxy, optionally substituted alkylcarbonyloxy, optionally substituted arylalkoxy, optionally substituted acyl, optionally substituted aminocarbonyl and carboxy.

Optionally, R₁, R₃, R₄ and R₅ are all H. Preferably, R₁, R₃, R₄ and R₅ are all H and R₂ is a group of the formula (II) above.

Optionally, R₈, R₁₁, R₁₂ are each H and R₉ and R₁₀ are each independently selected from halogen, optionally substituted alkyl, nitro, cyano, hydroxy, optionally substituted alkoxy, optionally substituted amino, carboxy, alkoxycarbonyl, methylenedioxy, ethylenedioxy, optionally substituted alkylcarbonyloxy and optionally substituted arylalkoxy.

Optionally, at least one of R₈, R₉, R₁₀, R₁₁, R₁₂ is selected from hydroxy and optionally substituted alkoxy. Optionally R₈, R₁₁, R₁₂ are each H and R₉ and R₁₀ are each independently selected from hydroxy and optionally substituted alkoxy. Optionally, R₈, R₉, R₁₁, R₁₂ are each H and R₁₀ is alkoxy, preferably methoxy or ethoxy.

In an embodiment, X is S, —Y is ═O, R₁, R₃, R₄ and R₅ are all H, and R₂ is of formula (II) above, wherein R₈, R₉, R₁₁, R₁₂ are each H and R₁₀ is methoxy.

In the formula (I) above, each alkyl may independently be a branched or straight chain alkyl, optionally a branched or straight chain C₁ to C₁₀ alkyl, optionally a branched or straight chain C₁ to C₆ alkyl, optionally a branched or straight chain C₁ to C₄ alkyl, optionally selected from methyl, ethyl, n-propyl, i-propyl, n-butyl, i-butyl and t-butyl. Each alkyl may be optionally substituted unless otherwise stated. A substituted alkyl is an alkyl that has one or more substituents. The substituents may be selected from halogen, nitro, cyano, hydroxy, optionally substituted alkoxy, optionally substituted amino, carboxy, alkoxycarbonyl, methylenedioxy, ethylenedioxy, optionally substituted alkylcarbonyloxy and optionally substituted arylalkoxy.

In the formula (I) above, each aryl may independently be selected from phenyl, naphthyl, dihydronaphthyl, tetrahydronaphthyl, indenyl and indanyl.

In the formula (I) above, each heteroaryl may independently be a mono- or bi-cyclic aromatic group having from 5 to 12 members and containing at least one hetero atom, optionally one, two or three heteroatoms, which, if more than one heteroatom is present, be the same or different. The at least one heteroatom may be selected from oxygen, nitrogen and sulphur. Each heteroaryl may independently be selected from thienyl, furyl, pyrrolyl, pyridyl and indolyl. Each aryl or heteroaryl may be substituted unless otherwise stated. A substituted aryl or heteroaryl is an aryl ring or heteroaryl ring having one or more substituents; optionally the substituents are selected from halogen, optionally substituted alkyl, nitro, cyano, hydroxy, optionally substituted alkoxy, optionally substituted amino, carboxy, alkoxycarbonyl, methylenedioxy, ethylenedioxy, optionally substituted alkylcarbonyloxy, optionally substituted arylalkoxy, optionally substituted acyl, optionally substituted aminocarbonyl and carboxy.

In formula (I) above, each halogen may be selected from fluorine, chlorine, bromine and iodine.

In formula (I) above, each optionally substituted amino may be selected from an unsubstituted amino group (—NH₂), an amino group substituted with one alkyl group and an amino group substituted with two alkyl groups.

The present invention provides pharmaceutically acceptable salts of compounds of formula (I). Pharmaceutically acceptable salts include addition salts, including salts formed with acids or bases. The acids may be selected from inorganic acids, for example hydrochloric, hydrobromic, nitric, sulphuric or phosphoric acids, phosphonic, or with organic acids, such as organic carboxylic acids, for example acetic, trifluoroacetic, lactic, succinic, glutaric, ascorbic, pyruvic, lactobionic, glycolic, oxalic, maleic, hydroxymaleic, fumaric, malic, malonic, tartaric, citric, salicylic, o-acetoxybenzoic, or organic sulphonic, 2-hydroxyethane sulphonic, toluene-p-sulphonic, methanesulphonic, camphoric, bisethanesulphonic acid or methanesulphonic acid. The bases may be selected from sodium hydroxide, potassium hydroxide, triethylamine, tert-butylamine.

The present invention provides prodrugs of compounds of formula (I). As will be understood by the skilled person, some of the compounds useful for the methods of the present invention may be available as prodrugs. As used herein, the term “prodrug” refers to a compound of formula (I) which has been structurally modified such that in vivo the prodrug is converted, for example, by hydrolytic, oxidative, reductive, or enzymatic cleavage, into the parent molecule (“drug”) as given by formula (I). Such prodrugs may be, for example, metabolically labile ester derivatives of the parent compound where said parent molecule bears a carboxylic acid group. Conventional procedures for the selection and preparation of suitable prodrugs are well known to one of ordinary skill in the art.

In an aspect, the present invention provides a method of activating a latent virus in a subject comprising
administering a compound of formula (I), or pharmaceutically acceptable salt thereof or prodrug thereof, to the subject. Formula (I) is as described herein.

In an aspect, the present invention provides a method of treating a subject having a latent virus comprising
administering a compound of formula (I), or pharmaceutically acceptable salt thereof or prodrug thereof, and an anti-viral agent to the subject. Formula (I) is as described herein.

In an aspect, the present invention provides an anti-microtubule agent for use in a method of treating a subject having a latent virus, the method comprising:
administering a compound of formula (I), or pharmaceutically acceptable salt thereof or prodrug thereof, and an anti-viral agent to the subject. Formula (I) is as described herein.

In an aspect, the present invention provides a composition comprising a compound of formula (I), or pharmaceutically acceptable salt thereof or prodrug thereof, and an anti-viral agent.

In an aspect, the present invention provides a kit, the kit comprising:
a compound of formula (I), or pharmaceutically acceptable salt thereof or prodrug thereof,
an anti-viral agent, and
instructions on how to treat the virus using the compound of formula (I), or pharmaceutically acceptable salt thereof or prodrug thereof, and the anti-viral agent. Formula (I) is as described herein.

Typically, the anti-microtubule agent is administered in an effective amount to activate the latent virus within a subject. The anti-microtubule agent may be administered alone or in a composition as described below. An effective amount of a microtubule agent that reactivates the latent virus, includes, but is not limited to, an amount that reactivates latent virus and reduces the reservoir of latent virus in an individual by at least about 20%, at least about 30%, at least about 40%, at least about 50%, at least about 60%, at least about 70%, at least about 80%, or at least about 90%. A “reduction in the reservoir of latent virus” (also referred to as “reservoir of latently infected cells”) is a reduction in the number of cells in the subject that harbor a latent virus infection. Whether the reservoir of latently infected cells is reduced can be determined using any known method, including, for HIV, the method described in Blankson et al. (2000) J. Infect. Disease 182(6):1636-1642.

In some embodiments, reactivation of latent virus in a cell results in death of the cell. Thus, in some embodiments, an effective amount of a subject agent that reactivates latent virus is an amount of a subject agent that reactivates latent virus is an amount that kills 10² or more, optionally 5×10² or more, optionally 10³ or more, optionally 5×10³ or more, optionally 10⁴ or more, optionally 5×10⁴ or more, optionally 1×10⁵ or more cells in an individual, which cells harbour the latent virus.

In the first aspect, the anti-microtubule agent may be administered alone or in a composition comprising one or more pharmaceutically acceptable carriers, diluents or excipients. Optionally, the anti-microtubule agent is administered with an anti-viral agent.

In the second aspect, the anti-microtubule agent may be administered to a subject at the same time as or at a different time as the anti-viral agent. Optionally, if the anti-microtubule agent and the anti-viral agent are administered separately, the anti-microtubule agent and the anti-viral agent are administered within about 5 minutes of each other, optionally, within about 10 minutes of each other, optionally within about 20 minutes of each other, optionally within about 30 minutes of each other, optionally within about 40 minutes of each other, optionally within about 50 minutes of each other, optionally within about 1 hour of each other, optionally within about 2 hours of each other, optionally within about 4 hours of each other, optionally within about 6 hours of each other, optionally within about 8 hours of each other, optionally within about 12 hours of each other, optionally within about 24 hours of each other, optionally within about 36 hours of each other. The anti-viral compound may be administered before, during or after activation of the virus.

The anti-viral agent may be any agent that can treat a subject having a virus in an active state. The anti-viral agent may be an anti-viral agent for treating a virus selected from a retrovirus and a herpes virus. The anti-viral agent may be an anti-viral agent for treating a retrovirus selected from the human immunodeficiency virus type I (HIV-1), human immunodeficiency virus type II (HIV-2), and Human T-lymphotropic virus Type I and II (HTLV-1, -2). The anti-viral agent may be an anti-viral agent for treating a herpes virus selected from herpes simplex virus-1, herpes simplex virus-2, varicella zoster virus, Epstein-Barr virus, cytomegalovirus, roseolovirus, Kaposi's sarcoma-associated herpesvirus, and all animal herpesviruses including, but not limited to, Equine herpesvirus, Marek's Disease virus, Porcine herpesvirus and Porcine cytomegalovirus.

The anti-viral agent may be an agent for treating HIV. The anti-viral agent may be an anti-retroviral agent. The anti-viral agent may be selected from nucleoside reverse transcriptase inhibitors, non-nucleoside reverse transcriptase inhibitors, nucleotide reverse transcriptase inhibitors, protease inhibitors, maturation inhibitors, attachment inhibitors, fusion inhibitors, entry inhibitors, integrase inhibitors, zinc finger inhibitors, chemokine receptor blockers and antisense molecules, and combinations thereof.

The nucleoside reverse transcriptase inhibitors may be selected from zidovudine, didanosine, zalcitabine, stavudine, lamivudine, abacavir, emtricitabine, entecavir, and apricitabine, and combinations thereof.

The non-nucleoside reverse transcriptase inhibitors may be selected from efavirenz, nevirapine, delavirdine, etravirine, and combinations thereof.

The nucleotide reverse transcriptase inhibitors may be selected from tenofovir and adefovir, and combinations thereof.

The protease inhibitors may be selected from saquinavir, ritonavir, indinavir, nelfinavir, amprenavir, lopinavir, atazanavir, fosamprenavir, tipranavir and darunavir, and combinations thereof.

The fusion inhibitors may be selected from maraviroc and enfuvirtide, and combinations thereof.

The integrase inhibitors may be selected from raltegravir and elvitegravir, and combinations thereof.

The agent for treating HIV may be a HAART (highly active antiretroviral therapy) agent, which, in the present application, includes a combination of at least two anti-retroviral agents, optionally at least three anti-retroviral agents. For example the HAART agent may comprise two different nucleoside reverse transcriptase inhibitors and one or more further anti-retroviral agents selected from, for example, a protease inhibitor, a non-nucleoside reverse transcriptase inhibitors and an integrase inhibitor. The HAART agent may be selected from, for example, (i) the combination of emtricitabine, tenofovir and efavirenz; (ii) the combination of emtricitabine, tenofovir and raltegravir; (iii) the combination of emtricitabine, tenofovir, ritonavir and darunavir; and (iv) the combination of emtricitabine, tenofovir, ritonavir and atazanavir.

The anti-viral agent may be an agent for treating a herpes virus. The agent for treating a herpes virus may be a DNA synthesis inhibitor. The agent for treating a herpes virus may be selected from a purine analog and a pyrimidine analogue. The agent for treating a herpes virus may be selected from acyclovir, valaciclovir, ganciclovir, valganciclovir, penciclovir, famciclovir, vidarabine, cytarabine, idoxuridine, trifluridine, edoxudine, brivudine, foscarnet, docosanol, fomivirsen, tromantadine, maribavir (5,6-dichloro-2-(isopropylamino)-1, beta-L-ribofuranosyl-1-H-benzimidazole) and AIC246 (C₂₉H₂₈F₄N₄O₄). AIC246 is an antiviral described in many publications, including Antimicrobial Agents and Chemotherapy, March 2010, p. 1290-1297, Vol. 54, No. 3, which is incorporated herein by reference in its entirety.

The present invention provides a composition comprising the anti-microtubule agent The composition may comprise the anti-microtubule agent and one or more pharmaceutically acceptable carriers, diluents or excipients, and optionally the anti-viral agent and/or one or more further agents for the activation of a latent virus.

The methods of the first, second and third aspects may comprise administering to a subject an anti-microtubule agent, which may be an anti-microtubule agent of formula (I), and a further agent for activating a latent virus. The further agent for activating a latent virus may not be an anti-microtubule agent. The anti-microtubule agent may be administered to a subject at the same time as or at a different time as the further agent for activating a latent virus. The composition may comprise anti-microtubule agent, which may be an anti-microtubule agent of formula (I), and one or more further agents for activating a latent virus. The one or more further agents for activating a latent virus may be selected from protein kinase C (PKC) activators (such as prostratin, jatrophane diterpene (SJ23B), 12-deoxyphorbol 13-phenylacetate(dPP)), DPP (12-deoxyphorbol 13-phenylacetate), an NF-κB inducer, an inhibitor of histone deacetaylase (which may, for example, be selected from valproic acid, suberoylanilide hydroxamic acid (SAHA), vorinostat and romidepsin), positive transcription elongation factor b (p-TEFb) activators (such as hexamethylene bisacetamide (HMBA)), an agent that cross-links cell-surface T-cell receptor, e.g. an anti-CD3 antibody. The NF-κB inducer may be selected from 5-hydroxynaphthalene-1,4-dione (HN), phytohemagglutinin (PHA), phorbol esters, e.g., tetradecatloyl phorbol acetate (TPA) and 4-α-phorbol 12-myristate 13-acetate (PMA); TNF-α; TNF-β; IL-1β; lipopolysaccharide; CD3 antibodies; a combination of CD3 and CD28 antibodies; Etopiside; Daunorubicin; hydrogen peroxide; Nocodazole; bleomycin; camptothecin; cisplatin; celecoxib; ciprofibrate; cycloprodigiosin; dacarbazine; Daio-Orengedeokuto; daunomycin; diazoxide; diclofenac; 5,6-dimethylxanthenone-4-acetic acid; flavone-8-acetic acid; haloperidol; imiquimod; isochamaejasmin; Kunbi-Boshin-Hangam-Tang (Koo et al, 2001, Immunopharmacol Immunotoxicol 23(2): 175-86); lithium (Nemeth et al, 2002, J Biol Chem 277(10):7713-9); mitoxantrone; morphine; nipradilol; norepinephrine; nystatin; oltipraz; protocatechuic acid; SN38; and tamoxifen. The further agent for activating a latent virus may be an agent for activating a virus described herein. The further agent for activating a latent virus may be an agent for activating HIV. The agent for activating HIV may be as described in WO 2007/121429, particularly paragraphs [0090] to [0105]. WO 2007/121429 is incorporated herein by reference in its entirety.

The methods of the first, second and third aspects may comprise administering to a subject an anti-microtubule agent, which may be an anti-microtubule agent of formula (I), and a further agent for activating a latent virus, wherein the further agent is selected from histone deacetylase inhibitors (which may, for example, be selected from valproic acid (VPA), suberoylanilide hydroxamic acid (SAHA), vorinostat and romidepsin), protein kinase C activators (such as prostratin, jatrophane diterpene (SJ23B) and 12-deoxyphorbol 13-phenylacetate(dPP)), NFkB activators (such as 5-hydroxynaphthalene-1,4-dione (HN)) and p-TEFb activators (such as hexamethylene bisacetamide (HMBA)). Administering the further latent virus-activating agents with the antimicrotubule agent, has been found to enhance the magnitude and breadth of the reactivation of latent viruses in different cell reservoirs. The anti-microtubule agent of formula (I) may be any of the embodiments described herein.

The methods of the first, second and third aspects may comprise administering to a subject an anti-microtubule agent of formula (I), and a further agent for activating a latent virus, wherein, in formula (I) X is S, —Y is ═O, R₁, R₃, R₄ and R₅ are all H, and R₂ is of formula (II) above, wherein R₈, R₉, R₁₁, R₁₂ are each H and R₁₀ is methoxy, and the further agent for activating a latent virus is selected from histone deacetylase inhibitors (such as valproic acid, suberoylanilide hydroxamic acid (SAHA), vorinostat and romidepsin), protein kinase C activators (such as prostratin, jatrophane diterpene (SJ23B) and 12-deoxyphorbol 13-phenylacetate(dPP)), NFkB activators (such as 5-hydroxynaphthalene-1,4-dione (HN)) and p-TEFb activators (such as hexamethylene bisacetamide (HMBA)). The anti-microtubule agent may be administered to a subject at the same time as or at a different time as the further agent for activating a latent virus. The composition may comprise anti-microtubule agent of formula (I), and one or more further agents for activating a latent virus, wherein, in formula (I) X is S, —Y is ═O, R₁, R₃, R₄ and R₅ are all H, and R₂ is of formula (II) above, wherein R₈, R₉, R₁₁, R₁₂ are each H and R₁₀ is methoxy, and the further agent for activating a latent virus is selected from histone deacetylase inhibitors (such as valproic acid, suberoylanilide hydroxamic acid (SAHA), vorinostat and romidepsin), protein kinase C activators (such as prostratin, jatrophane diterpene (SJ23B) and 12-deoxyphorbol 13-phenylacetate(dPP)), NFkB activators (such as 5-hydroxynaphthalene-1,4-dione (HN)) and p-TEFb activators (such as hexamethylene bisacetamide (HMBA)).

Optionally, the one or more further agents for activating a latent virus may be selected from anti-microtubule agents, for example as described herein. The composition and kit may comprise two or more anti-microtubule agents, optionally the anti-viral agent, and one or more pharmaceutically acceptable carriers, diluents or excipients. For example, the composition may comprise a first anti-microtubule agent and a second anti-microtubule agent, wherein the first and second anti-microtubule agents are different from one another. Each of first and second anti-microtubule agents may independently be selected from a compound of formula (I), genistein, vinca alkaloids (including, but not limited to, vincristine, vinblastine, vindesine and vinorelbin), taxotere, maytansin, rhizoxin, taxane compounds, and combinations thereof. In an embodiment, the composition may comprise an anti-microtubule agent of formula (I) and one or more agents selected from prostratin, genistein, vinca alkaloids (including, but not limited to, vincristine, vinblastine, vindesine and vinorelbin), taxotere, maytansin, rhizoxin, taxane compounds, and combinations thereof.

The composition may be formulated for clinical use and the formulation will vary according to the particular type of subject and the agent(s) being adminstered. The subject may be a human or animal, for example a human or non-human mammal. The dosage amounts and frequency of administration will also vary according to the formulation, and type of subject. Generally, determining dosage forms, dosage amount and frequency can be accomplished using conventional pharmacological formulations, clinical dosing studies, coupled with appropriate diagnostics.

The pharmaceutical composition may be a composition for injection. Such pharmaceutical compositions can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol. The composition may further comprise conventional additives such as solubilizers, isotonic agents, suspending agents, emulsifying agents, stabilizers and preservatives. If the composition is in the form of an aqueous solutions, the composition may comprise physiologically compatible buffers such as Hanks' solution, Ringer's solution, or physiological saline buffer. For transmucosal administration, penetrants appropriate to the barrier to be permeated are used in the formulation. Such penetrants are generally known in the art.

The composition may be a composition for oral administration. Such pharmaceutical compositions can be formulated readily by combining with pharmaceutically acceptable carriers that are well known in the art. The composition may be in a form including, but not limited to, tablets, pills, dragees, capsules, emulsions, lipophilic and hydrophilic suspensions, liquids, gels, syrups, slurries and suspensions. A composition for oral administration can be obtained by mixing the active ingredients (e.g. the anti-microtubule agent and/or an anti-viral agent to the subject) with a solid excipient, optionally grinding the resulting mixture, and processing the mixture of granules, after adding suitable auxiliaries, if desired, to obtain tablets or dragee cores. Suitable excipients include, but are not limited to, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

The composition may further comprise antioxidants or preservatives. Antioxidants include, but are not limited to, sodium sodium sulphite, sodium hydrogen sulphite, sodium metabisulphite, ascorbic acid, ascorbylpalmitate, -myristate, -stearate, gallic acid, gallic acid alkyl ester, butylhydroxyamisol, nordihydroguaiaretic acid, tocopherols as well as synergists (substances which bind heavy metals through complex formation, for example lecithin, ascorbic acid, phosphoric acid ethylene diamine tetracetic acid, citrates, tartrates). Addition of synergists can increase the antioxygenic effect of the antioxidants.

The composition may comprise a preservative. Preservatives include, but are not limited to, sorbic acid, p-hydroxybenzoic acid esters (for example lower alkyl esters), benzoic acid, sodium benzoate, trichloroisobutyl alcohol, phenol, cresol, benzethonium chloride, chlorhexidine and formalin derivatives.

The composition may be in the form of a dragee, i.e. a core containing an active having a coating, typically a sugar coating, thereon. The sugar coating may be formed from a sugar solution that may further comprise gum arabic, talc, polyvinyl pyrrolidone, carbopol gel, polyethylene glycol, and/or titanium dioxide, lacquer solutions, dyestuffs, pigments, and suitable organic solvents or solvent mixtures.

The composition may be in the form of a capsule containing the active ingredients. The capsule may, for example, be a push-fit capsule made of gelatin, as well as soft, sealed capsules made of gelatin and a plasticizer, such as glycerol or sorbitol. The capsules can contain the active ingredients, e.g. the anti-microtubule agent and, optionally, an anti-viral agent to the subject, in admixture with a filler, such as lactose, a binder such as starch, and/or a lubricant such as talc or magnesium sterate and, optionally, stabilizers. In soft capsules, the active ingredients may be dissolved or suspended in suitable liquids, such as fatty oils, liquid paraffin, or liquid polyethylene glycols. Optionally, the capsules may further contain a stabilizer.

The composition may be in a form for buccal administration. For example, the pharmaceutical composition may be in the form of tablets or lozenges suitable for this purpose.

The composition may be in a form for administration by inhalation. The composition may, for example, be administered in the form of an aerosol spray from a suitable container, e.g. from pressurized container.

The composition may be in a form for administration parenterally. Such compositions may be suspension, solutions or emulsions in oily or aqueous liquids.

The compositions may be in a form for rectal administration, for example in a form such as a suppository or retention enemas. Optionally, such compositions may contain conventional suppository bases such as cocoa butter, carbowaxes, polyethylene glycols or other glycerides, all of which melt at body temperature, yet are solidified at room temperature.

The composition may comprise one or more liposomes for liposomal drug delivery. Liposomes and emulsions are well known examples of delivery vehicles or carriers for hydrophobic drugs.

The composition may be a composition for topical and/or subcutaneous administration. For example the composition may be in the form of a gel or cream for topical and/or subcutaneous administration. The composition may include one or more carriers for topical administration of the anti-microtubule agent and/or anti-viral agent, and any other active agents that may be present in the composition. The carriers for topical administration may include, but are not limited to, mineral oil, liquid petroleum, white petroleum, propylene glycol, polyoxyethylene polyoxypropylene compound, emulsifying wax and water. In an embodiment, the pharmaceutical composition can be formulated with a suitable lotion or cream containing the active compound suspended or dissolved in a carrier. Suitable carriers for a lotion or cream include, but are not limited to, mineral oil, sorbitan monostearate, polysorbate 60, cetyl esters wax, cetearyl alcohol, 2-octyldodecanol, benzyl alcohol and water.

The compositions may contain a suitable dosage amount of the anti-microtubule agent and, if present, the anti-viral agent and/or the one or more further agents for activating a latent virus. The anti-microtubule agent and, if present, the anti-viral agent and/or the one or more further agents for activating a latent virus, may each independently be present in an amount suitable for the nature of the subject being administered and the virus. The anti-microtubule agent may be present in the composition in an amount of, for example, 0.01 to 100 mg, e.g. from 0.1 to 5 mg, e.g. 0.1 to 5 mg. The anti-viral agent and/or the one or more further agents for activating a latent virus may be present in the composition in an amount of, for example, 0.01 to 100 mg, e.g. from 0.1 to 5 mg, e.g. 0.1 to 5 mg. The composition may administered as often as necessary, for example one, two or three times a day.

The composition may further comprise one or more therapeutic agents, other than the anti-microtubule agent and/or the anti-viral agent and/or the one or more further agents for activating a latent virus, including, but not limited to, an agent selected from anti-inflammatory agents, anti-fungal agents, anti-bacterial agents, ameobicidal agents, analgesic agents, anti-neoplastic agents, anti-hypertensive agents.

In a fifth aspect, the present invention provides a kit, the kit comprising:
an anti-microtubule agent,
an anti-viral agent, and
instructions on how to treat the virus using the anti-microtubule agent, and anti-viral agent. The kit may further comprise a further agent for activating a latent virus, which may be as described herein.

The anti-microtubule agent may be packaged separately from the anti-viral agent and, if present, the further agent for activating a latent virus, e.g. in different packaged formulations or packaged together, e.g. in the same formulation. The kit may comprise a package containing one or more dosage forms of a composition comprising the anti-microtubule agent and one or more dosage forms of a composition comprising the anti-viral agent, and optionally one or more dosage forms of a composition comprising one or more further agents for activating a latent virus.

The instructions on how to treat the virus using the anti-microtubule agent and the anti-viral agent, and, if present, the further agent for activating a latent virus, may comprise one or more sheets, e.g. paper sheets, with written instructions thereon or a computer readable medium having the instructions stored thereon.

The present invention will now be described by way of example only with reference to the following non-limiting Examples.

### EXAMPLES

### Example 1

### Identification of Small Molecules which Induce the KSHV Lytic Cycle

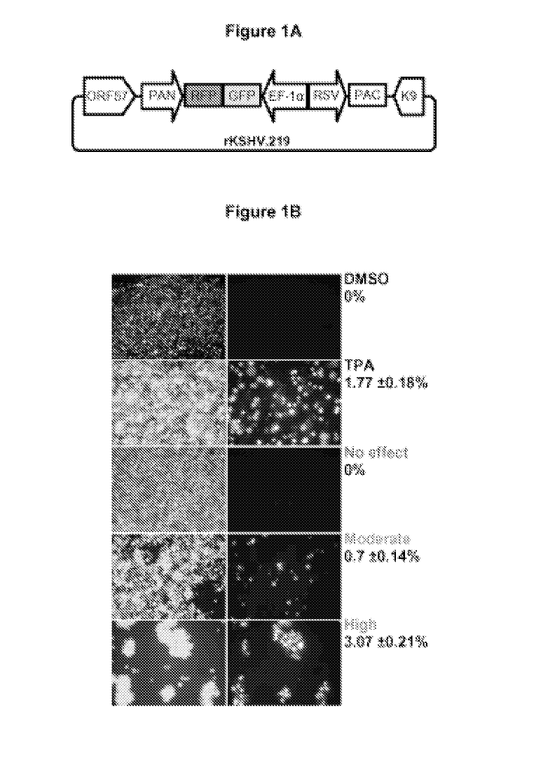

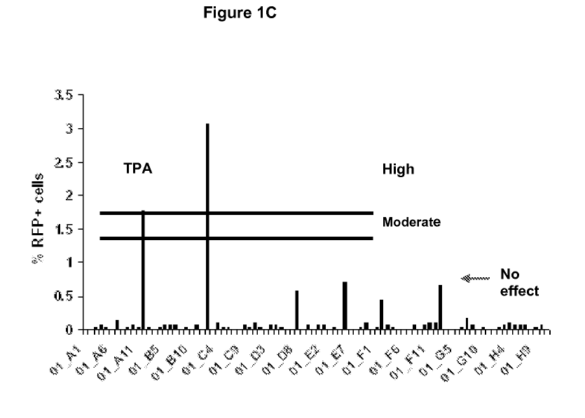

To identify novel host proteins that induce KSHV reactivation, the present inventors utilized a primary effusion lymphoma (PEL) cell-line (JSC-1) that is infected with a dual-fluorescent recombinant KSHV (rKSHV.219)¹⁶. The present inventors screened the NCI DTP Diversity set (1990 compounds; http://dtp.cancer.gov) for inducers of the KSHV lytic switch RTA (replication and transcription activator or ORF 50), as viral protein RTA is necessary and sufficient to drive latent KSHV into lytic cycle¹⁷. PEL cells which harbor latent KSHV and ˜80% are latently co-infected with EBV (see review¹⁸. JSC-1 rKSHV.219 cells are thus naturally infected with latent KSHV and EBV, and superinfected with latent rKSHV.219. rKSHV.219 encodes green fluorescent protein (GFP) under the control of an EF1-α promoter and red fluorescent protein (RFP) under the control of the KSHV lytic PAN promoter (FIG. 1A). All latently infected cells therefore uniformly express GFP and less than 0.1% of cells express detectable levels of RFP representing spontaneous lytic replication. Following lytic reactivation by 12-O-tetradecanoyl-phorbol-13-acetate (TPA), KSHV RTA transactivates the PAN promoter inducing strong RFP expression (FIG. 1B). Any compounds that induced 0.3% or higher RFP-expressing cells are considered positive [three times above the dimethyl sulfoxide (DMSO) control at 0.1% RFP-expressing cells] in the primary screen. In total we identified 70 out of 1990 compounds able to induce RFP expression and these primary hits were classified, with respect to the TPA control (−1.8%), into hits producing moderate (0.3-0.9%-61 hits) or high levels (>0.9%-9 hits) of reactivation (an example in FIG. 1C).

### Example 2

### KSHV Induction Kinetics by Active Compounds

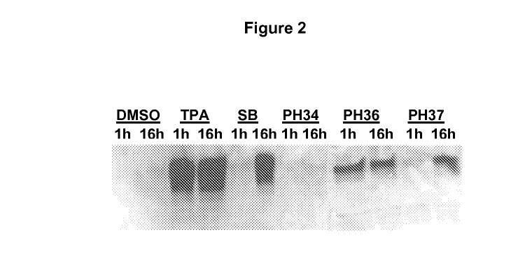

The present inventors had previously noted that conventional chemical stimuli for reactivating KSHV such as TPA (a phorbol ester) and sodium butyrate (SB, a HDAC inhibitor) work with different induction kinetics. TPA requires short exposure (immediate-kinetics; <1 hour) while SB requires longer exposure time (delayed-kinetics) (FIG. 2). TPA induces signal transduction cascades leading to KSHV lytic cycle through the activation of PKC. Once signal is initiated TPA is no longer required and hence the immediate-kinetics. Therefore, compounds acting with immediate-kinetics may also induce signal transduction pathways. We therefore characterized the induction kinetics of the 70 primary hits. This also allowed us to verify the ability of these compounds to induce KSHV RTA protein expression. Of the 70 primary hits tested 45 failed to induce RTA, for example, PH34 (Primary Hit 34). Two hits were similar to the TPA control, for example, PH36. Twenty three hits were akin to the SB control, for example, PH37 (FIG. 2).

### Example 3

### UCLB-15026 Reactivates Latent KSHV Through Disruption of Microtubule Dynamics, and UCLB-15026 Sensitizes PEL to Ganciclovir

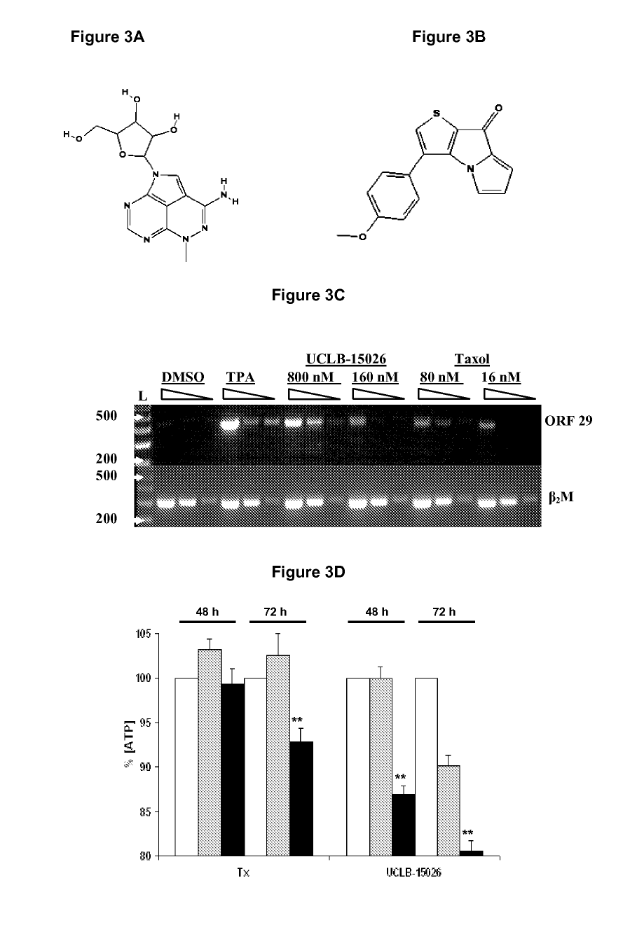

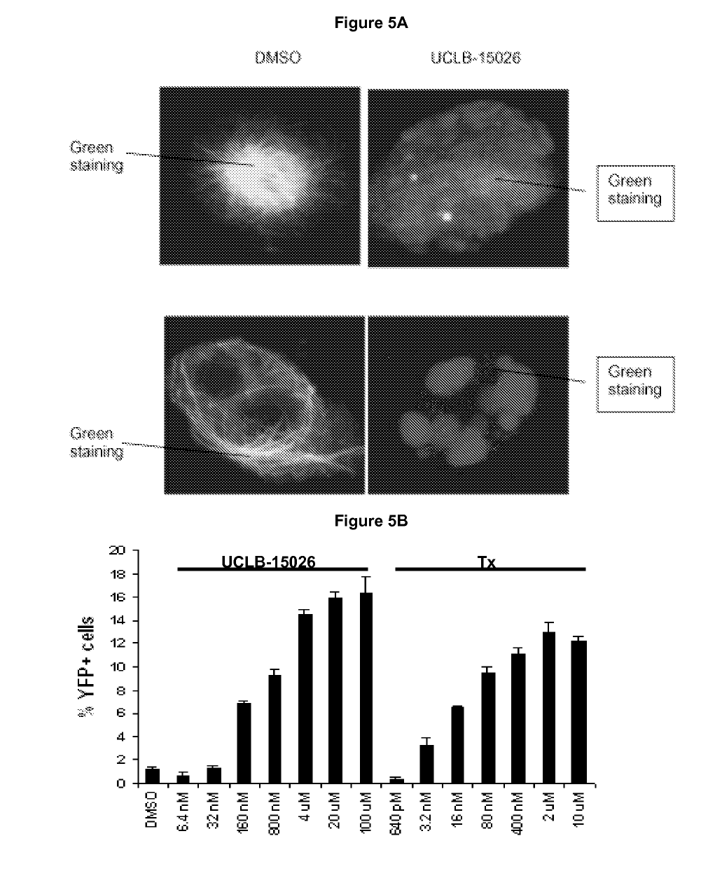

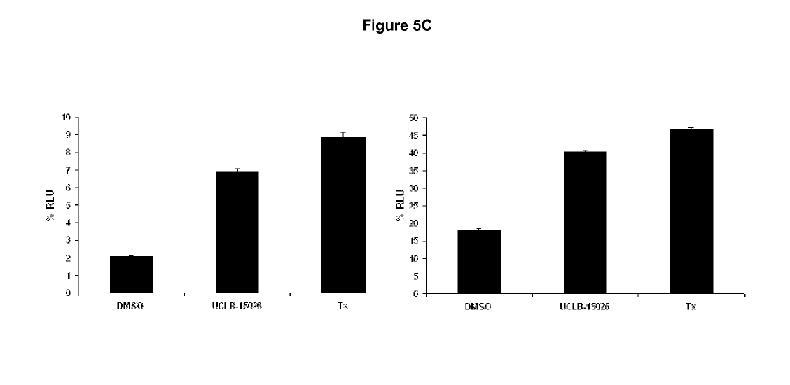

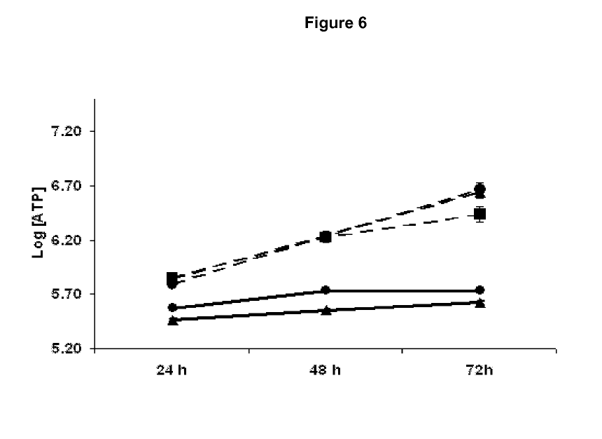

The majority of the 25 validated primary hits have no known cellular targets with two exceptions, PH30 and PH36. PH30 (FIG. 3A), or Triciribine, is a highly selective inhibitor of the Akt pathway¹⁹. Inhibition of the Akt pathway induces KSHV lytic cycle²⁰, thus supporting this approach for the discovery of small molecules that reactivate KSHV. PH36 (FIG. 3B; hereafter referred to as UCLB-15026) has been reported to be an anti-microtubule agent inhibiting tubulin polymerization (acting as a tubulin destabilizer) with an IC₅₀ of 2.9 μM²¹. Cells treated with the compound accumulated in the G₂/M phases of cell cycle and displayed increased nuclear DNA content resulting from reinitiation of DNA synthesis cycle during cell cycle arrest—a hallmark of cells perturbed with anti-microtubule agents. Immunofluorescence microscopy of JSC-1 cells after treatment with UCLB-15026 verified that it disrupts microtubule network in KSHV-infected PEL cells (FIG. 5A). To confirm that interfering with microtubule dynamics induces KSHV lytic reactivation, the present inventors treated JSC-1 cells with another known anti-microtubule agent paclitaxel (taxol; a tubulin stabilizer). Both UCLB-15026 and taxol reactivated KSHV to similar levels and displayed half their maximal activities in the nanomolar range (FIG. 5B). Both compounds also induced RTA in BCBL-1 cells, a PEL cell line only infected with KSHV, showing that KSHV reactivation is independent of EBV (FIG. 5C). Both compounds induced a dose-dependent expression of a KSHV late gene ORF 29 in JSC-1 (FIG. 5C), confirming the induction of full virus lytic cycle. Induction of the KSHV lytic cycle should render PEL cells susceptible to antiviral-mediated cytotoxicity of ganciclovir (GCV). GCV alone had no detrimental effect on JSC-1 proliferation (FIG. 6). Pre-treatment with UCLB-15026 or taxol arrested cell proliferation but had no obvious cytotoxicity over 72 hours, consistent with their mode of action. However, pre-treatment of JSC-1 cells with taxol for 24 h, followed by cell culture in the presence of GCV resulted in a small but statistically significant increase in cell cytotoxicity at 72 h with high-dose GCV (100 μM). Similarly, UCLB-15026 pre-treatment also enhanced GCV cytotoxicity against PEL cells (FIG. 3D) and GCV activities mirror levels of ORF 29 expression (FIG. 3C), demonstrating its potential as an antiviral/antitumor adjuvant.

### Example 4

### UCLB-15026 Activates LTR Transcriptional Activity in a HIV-1 Latency Model

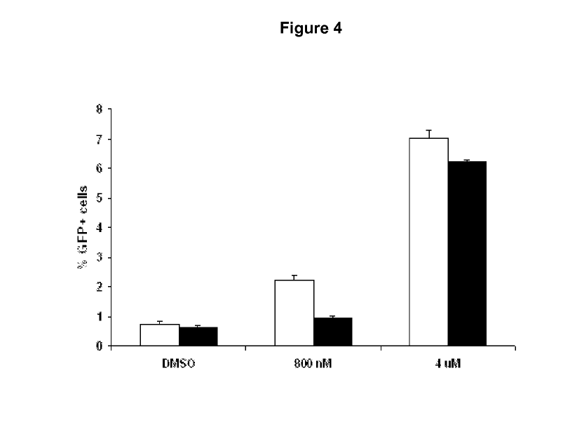

Reactivation stimuli that induce replicative cycle of latent virus often work across different virus families, for example, HDAC inhibitors and prostratin^{6,14,22,23}. The present inventors therefore examined whether UCLB-15026 can also activate latent HIV-1 using the J-Lat model^{24,25}. The J-Lat cell line A72 is a cell clone derived from Jurkat T cells harboring a transcriptionally inactive HIV-1 LTR-GFP construct. The present inventors found that treatment of J-Lat cells with UCLB-15026 substantially induced LTR-mediated GFP expression (FIG. 4). This demonstrates that UCLB-15026 increases transcription activity of both KSHV and HIV-1 in latency models.

### Example 5

### Broad Spectrum Antilatency Activity of Genistein

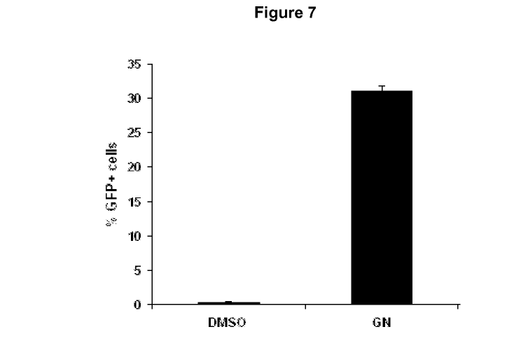

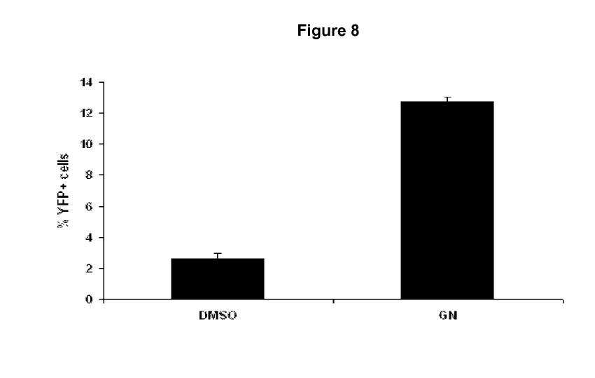

The present inventors demonstrated the induction of HIV-1 LTR activity in J-Lat A72 cells by genistein (see FIG. 7), and they also demonstrated for the first time that genistein can reactivate KSHV in PEL (see FIG. 8). Together the data show that compounds altering microtubule dynamics including UCLB-15026, taxol and genistein reactivate latent KSHV and HIV-1.

### Methods and Materials for Examples 1 to 5

Cell Culture.

All cell lines were grown in complete media consisted of RPMI medium 1640 (Invitrogen) with 10% FBS (BioSera) and 100 units/ml penicillin/streptomycin (Invitrogen) at 37° C. in 5% carbon dioxide. JSC-1 r219 cell-line was a kind gift from Jeff Vieira (University of Washington, Seattle, Wash.). BCBL-1 and J-LAT A72 cells were obtained through the NIH AIDS Research and Reference Reagent Program, Division of AIDS, NIAID, NIH.

Chemicals.

NCI DTP Diversity set and follow-up small molecules were obtained from the NCI/DTP Open Chemical Repository; all small molecules were reconstituted in DMSO to 10 mM. Other chemicals used include DMSO (Sigma), TPA (Merck), sodium butyrate (Merck), taxol (Sigma), ganciclovir (Roche), and genistein (Merck).

Primary Screen.

JSC-1 r219 cells were plated at approximately 2×10⁴ cells per well in 96-well plates and compounds were added to each well (100 μM final concentration). Levels of RFP expression of each sample were determined by FACSarray (BD Bioscience) at 48 h post-treatment.

Secondary Screen.

JSC-1 cells were plated at 4×10⁵ cells per well in 48-well plates and positive primary hits were added to each well (100 μM final concentration) and the compound treatment lasted for either 1 h or 16 h, and cells were harvested at 16 h post-treatment. Equal number of cells of each sample was lysed in sample buffer [Tris HCl (0.2 M), pH 6.8, 5.2% sodium dodecyl sulphate (SDS), 20% glycerol, and bromophenol blue]. The samples were resolved by 10% SDS-polyacrylamide gels and transferred to a polyvinylidene fluoride membrane (GE Healthcare). The membrane was blocked with nonfat dry milk in Tris-buffered saline containing 0.1% Tween 20 (TBST), followed by incubation with rabbit polyclonal anti-RTA (a kind gift from Don Ganem, UCSF;⁴¹ and then incubation with an appropriate secondary antibody. The membrane was washed, followed by signal development with ECL reagents (GE Healthcare).

RNA Extraction and Reverse Transcriptase PCR (RT-PCR).

Total RNA was purified and reverse transcribed as previously described¹⁵ with modification. Briefly, RNA was extracted from 2×10⁶ cells with TRIzol (Invitrogen) and treated with DNase (Ambion), followed by phenol extraction and ethanol precipitation. Serial dilution (3×10-fold) of oligonucleotide dT (Qiagen)-primed cDNA was used for PCR amplification (Promega) of KSHV ORF 29 using forward (GCA CAC ACG TAA CTG ACC AC; SEQ ID NO. 1) and reverse (CAT TGG GTA CGT AGC CCA CC; SEQ ID NO. 2) primers. All samples were normalized by PCR amplification of beta-2 microglobulin (β₂M) using serially diluted cDNA with forward (TGA CTT TGT CAC AGC CCA AG; SEQ ID NO. 3) and reverse (TCT CTG CTC CCC ACC TCT AA; SEQ ID NO. 4) primers. The PCR cycle consisting of 95° C. for 30 seconds, 50° C. for 30 seconds, and 72° C. for 30 seconds was repeated 36 times for ORF 29, and 28 times for β₂M. Both set of primers amplify across splice junction and the sizes (in bp) of the expected products are: ORF 29 (cDNA—399, DNA—3650) and β₂M (cDNA—301, DNA—2178).

Cell Viability Assay.

To test if UCLB-15026-induction sensitizes PEL to GCV killing, JSC-1 cells were pre-treated with UCLB-15026 or taxol for 24 h, and cultured in fresh media containing either no drug, or GCV at 20 μM or 100 μM. All samples were measured for cell viability at 48 and 72 h post media replacement using the CellTiter-Glo assay (Promega). For the compound control experiments, JSC-1 cells were treated with DMSO for 24 h, and cultured in fresh media containing no drug, or GCV at 20 μM or 100 μM. In addition, JSC-1 cells were treated with UCLB-15026 (800 nM) or taxol (80 nM) for 24 h and cultured in fresh drug free media. All samples were measured for viability at 24, 48, and 72 h (post media replacement) using the CellTiter-Glo assay.

HIV-1 LTR Assay.

Levels of GFP expression in J-LAT A72 cells were measured by FACScan following incubation with TPA (20 ng/ml), genistein (100 μM), or UCLB-15026 at different concentrations.

Confocal Microscopy.

JSC-1 cells were grown on coverslips coated with L-glycine (Sigma) with DMSO or UCLB-15026 (100 μM) for 16 h. JSC-1 cells were then fixed in 3% paraformaldehyde, rinsed and permeabilized in 0.02% Triton-PBS. This was followed by blocking in 2% FCS-PBS, staining with anti-tubulin antibody (Sigma), washed, and then incubated with Alexa-488 conjugated goat anti-mouse secondary antibody (Molecular Probes) before being mounted onto slides using VectaShield mounting medium containing DAPI. Images were taken using confocal microscope (Leica).

KSHV Lytic Cycle Induction Assays.

Induction levels were measured either by increased levels of yellow fluorescent protein (YFP)— or luciferase-expression. JSC-1 stably transduced with a KSHV PAN promoter-driven YFP reporter (PYFP), or JSC-1 and BCBL-1 cells stably transduced with a KSHV PAN promoter-driven luciferase reporter (PLuc) were established and all these reporter cell-lines express increasing amounts of either YFP or luciferase in response to KSHV RTA induction (E Tsao & P Kellam, manuscript in preparation). JSC-1 PYFP reporter cells were used for the titration of UCLB-15026 or taxol, and also for studying the effect of genistein. Levels of YFP expression were measured by FACSarray (BD Bioscience). JSC-1 PLuc and BCBL-1 PLuc cells were cultured with either UCLB-15026 (800 nM) or taxol (80 nM), and at 40 h post-treatment, the total number of cells of each sample was counted before cell lysis. The relative levels of RTA induction between different lysates were determined by measuring relative light units (RLU) using the Promega Bright-glo kit and a GloMax 96-microplate luminometer (Promega) as per the manufacturer's instructions. The luciferase activity was reported as % activity normalized to the positive control (TPA) at an input of 1×10⁵ cells.

In summary, the present inventors have identified anti-microtubule compounds that interfere with microtubule dynamics and reactivate KSHV and HIV-1 in latency models. This demonstrates that anti-microtubule compounds possess broad spectrum activity against latent viruses and can be used for adjunctive antiviral therapy.

References mentioned herein or otherwise useful for background:
1. Boshoff, C. & Weiss, R. AIDS-related malignancies. Nat Rev Cancer 2, 373-82 (2002).
2. Siliciano, J. D. et al. Long-term follow-up studies confirm the stability of the latent reservoir for HIV-1 in resting CD4+ T cells. Nat Med 9, 727-8 (2003).
3. Strain, M. C. et al. Heterogeneous clearance rates of long-lived lymphocytes infected with HIV: intrinsic stability predicts lifelong persistence. Proc Natl Acad Sci USA 100, 4819-24 (2003).
4. Gane, E. et al. Randomised trial of efficacy and safety of oral ganciclovir in the prevention of cytomegalovirus disease in liver-transplant recipients. The Oral Ganciclovir International Transplantation Study Group [corrected]. Lancet 350, 1729-33 (1997).
5. Peterson, S., Reid, A. P., Kim, S. & Siliciano, R. F. Treatment implications of the latent reservoir for HIV-1. Adv Pharmacol 55, 411-25 (2007).
6. Lehrman, G. et al. Depletion of latent HIV-1 infection in vivo: a proof-of-concept study. Lancet 366, 549-55 (2005).
7. Feng, W. H. & Kenney, S. C. Valproic acid enhances the efficacy of chemotherapy in EBV-positive tumors by increasing lytic viral gene expression. Cancer Res 66, 8762-9 (2006).
8. Klass, C. M., Krug, L. T., Pozharskaya, V. P. & Offermann, M. K. The targeting of primary effusion lymphoma cells for apoptosis by inducing lytic replication of human herpesvirus 8 while blocking virus production. Blood 105, 4028-34 (2005).
9. Kellam, P. Attacking pathogens through their hosts. Genome Biol 7, 201 (2006).
10. del Real, G. et al. Statins inhibit HIV-1 infection by down-regulating Rho activity. J Exp Med 200, 541-7 (2004).
11. Brass, A. L. et al. Identification of host proteins required for HIV infection through a functional genomic screen. Science 319, 921-6 (2008).
12. Karlas, A. et al. Genome-wide RNAi screen identifies human host factors crucial for influenza virus replication. Nature 463, 818-22.
13. Krishnan, M. N. et al. RNA interference screen for human genes associated with West Nile virus infection. Nature 455, 242-5 (2008).
14. Ylisastigui, L., Archin, N. M., Lehrman, G., Bosch, R. J. & Margolis, D. M. Coaxing HIV-1 from resting CD4 T cells: histone deacetylase inhibition allows latent viral expression. Aids 18, 1101-8 (2004).
15. Wilson, S. J. et al. X box binding protein XBP-1s transactivates the Kaposi's sarcoma-associated herpesvirus (KSHV) ORF50 promoter, linking plasma cell differentiation to KSHV reactivation from latency. J Virol 81, 13578-86 (2007).
16. Vieira, J. & O'Hearn, P. M. Use of the red fluorescent protein as a marker of Kaposi's sarcoma-associated herpesvirus lytic gene expression. Virology 325, 225-40 (2004).
17. Sun, R. et al. A viral gene that activates lytic cycle expression of Kaposi's sarcoma-associated herpesvirus. Proc Natl Acad Sci USA 95, 10866-71 (1998).
18. Carbone, A., Cesarman, E., Gloghini, A. & Drexler, H. G. Understanding pathogenetic aspects and clinical presentation of primary effusion lymphoma through its derived cell lines. Aids 24, 479-90.
19. Yang, L. et al. Akt/protein kinase B signaling inhibitor-2, a selective small molecule inhibitor of Akt signaling with antitumor activity in cancer cells overexpressing Akt. Cancer Res 64, 4394-9 (2004).
20. Peng, L. et al. Inhibition of the phosphatidylinositol 3-kinase-Akt pathway enhances gamma-2 herpesvirus lytic replication and facilitates reactivation from latency. J Gen Virol 91, 463-9.
21. Lisowski, V. et al. Design, synthesis, and evaluation of novel thienopyrrolizinones as antitubulin agents. J Med Chem 47, 1448-64 (2004).
22. Kulkosky, J. et al. Prostratin: activation of latent HIV-1 expression suggests a potential inductive adjuvant therapy for HAART. Blood 98, 3006-15 (2001).
23. Brown, H. J., McBride, W. H., Zack, J. A. & Sun, R. Prostratin and bortezomib are novel inducers of latent Kaposi's sarcoma-associated herpesvirus. Antivir Ther 10, 745-51 (2005).
24. Jordan, A., Defechereux, P. & Verdin, E. The site of HIV-1 integration in the human genome determines basal transcriptional activity and response to Tat transactivation. Embo J 20, 1726-38 (2001).
25. Jordan, A., Bisgrove, D. & Verdin, E. HIV reproducibly establishes a latent infection after acute infection of T cells in vitro. Embo J 22, 1868-77 (2003).
26. Wang, T. H. et al. Microtubule-interfering agents activate c-Jun N-terminal kinase/stress-activated protein kinase through both Ras and apoptosis signal-regulating kinase pathways. J Biol Chem 273, 4928-36 (1998).
27. Gozlan, J., Lathey, J. L. & Spector, S. A. Human immunodeficiency virus type 1 induction mediated by genistein is linked to cell cycle arrest in G2. J Virol 72, 8174-80 (1998).
28. Thierry, S. et al. Cell cycle arrest in G2 induces human immunodeficiency virus type 1 transcriptional activation through histone acetylation and recruitment of CBP, NF-kappaB, and c-Jun to the long terminal repeat promoter. J Virol 78, 12198-206 (2004).
29. Jeening a, R. E., Westerhout, E. M., van Gerven, M. L. & Berkhout, B. HIV-1 latency in actively dividing human T cell lines. Retrovirology 5, 37 (2008).
30. Mukherjee, S., Acharya, B. R., Bhattacharyya, B. & Chakrabarti, G. Genistein arrests cell cycle progression of A549 cells at the G(2)/M phase and depolymerizes interphase microtubules through binding to a unique site of tubulin. Biochemistry 49, 1702-12.
31. Saville, M. W. et al. Treatment of HIV-associated Kaposi's sarcoma with paclitaxel. Lancet 346, 26-8 (1995).
32. Gill, P. S. et al. Paclitaxel is safe and effective in the treatment of advanced AIDS-related Kaposi's sarcoma. J Clin Oncol 17, 1876-83 (1999).
33. Solan, A. J., Greenwald, E. S. & Silvay, O. Long-term complete remissions of Kaposi's sarcoma with vinblastine therapy. Cancer 47, 637-9 (1981).
34. Odom, R. B. & Goette, D. K. Treatment of cutaneous Kaposi's sarcoma with intralesional vincristine. Arch Dermatol 114, 1693-4 (1978).
35. Sgadari, C. et al. Mechanism of paclitaxel activity in Kaposi's sarcoma. J Immunol 165, 509-17 (2000).
36. Antman, K. & Chang, Y. Kaposi's sarcoma. N Engl J Med 342, 1027-38 (2000).
37. An, J., Sun, Y., Fisher, M. & Rettig, M. B. Antitumor effects of bortezomib (PS-341) on primary effusion lymphomas. Leukemia 18, 1699-704 (2004).
38. Munoz-Fontela, C. et al. Induction of paclitaxel resistance by the Kaposi's sarcoma-associated herpesvirus latent protein LANA2. J Virol 82, 1518-25 (2008).
39. Geeraert, L., Kraus, G. & Pomerantz, R. J. Hide-and-seek: the challenge of viral persistence in HIV-1 infection. Annu Rev Med 59, 487-501 (2008).
40. Richman, D. D. et al. The challenge of finding a cure for HIV infection. Science 323, 1304-7 (2009).
41. Lukac, D. M., Renne, R., Kirshner, J. R. & Ganem, D. Reactivation of Kaposi's sarcoma-associated herpesvirus infection from latency by expression of the ORF 50 transactivator, a homolog of the EBV R protein. Virology 252, 304-12 (1998).

### Example 6

### Induction of KSHV Lytic Cycle by UCLB-15026 is Dependent on the ERK Pathway

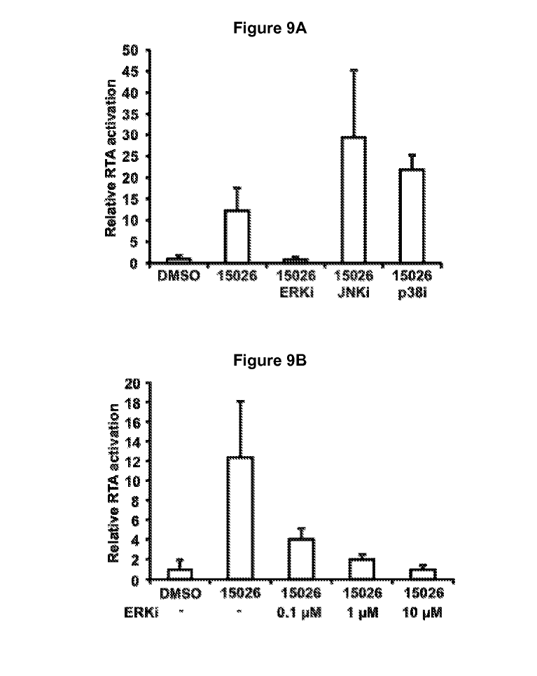

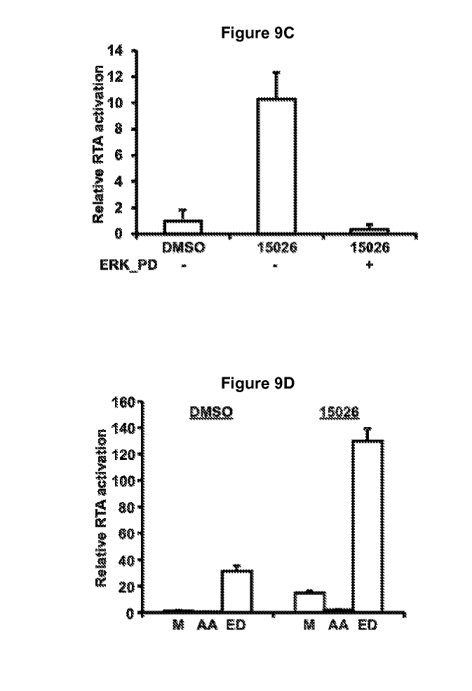

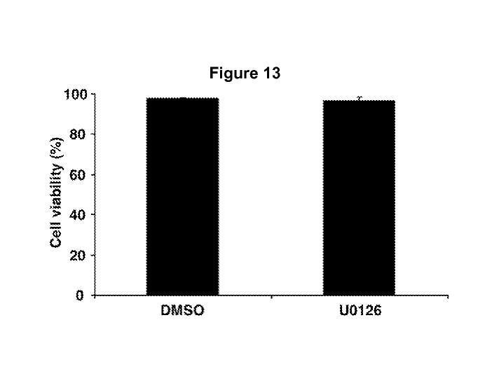

Interference with microtubule dynamics can result in caspase-mediated apoptosis and/or modulation of Mitogen-activated protein kinases (MAPKs)^{42,43}. To determine whether 15026 operated through inducing apoptosis, we treated JSC-1 cells with the pan-caspase inhibitor Q-VD-OPH and showed blocking apoptosis (FIG. 12A) had no effect on KSHV reactivation mediated by 15026 (FIG. 12B). We next tested whether the effects of 15026 are mediated through the MAPK pathways. The three major MAPK sub-pathways include ERK, p38 and c-Jun N-terminal kinase (JNK) pathways were examined using pathway-specific kinase inhibitors. Their use for studying MAPK pathways and KSHV in PEL cells is well established^{44,45}. We found that treatment of JSC-1 cells with SB203580 (p38 inhibitor) or SP600125 (JNK inhibitor) had no inhibitory effect (FIG. 9A). In contrast, treatment with U0126 (ERK inhibitor) led to reduced levels of RTA induction (FIG. 9A) by 15026, and the inhibition was dose-dependent (FIG. 9B). We confirmed by trypan blue exclusion test that treatment of PEL cells with U0126 had no significant effect on cell viability (FIG. 13). In addition, RTA induction by 15026 was inhibited by a different ERK-specific inhibitor PD0325901 (FIG. 9C). Furthermore, ectopic expression of a MAPK kinase 1 (MEK1) mutant (MEK-ED) that constitutively phosphorylates ERK⁴⁶ potentiated RTA induction by 15026 (FIG. 9D). In contrast, ecotopic expression of a dominant negative MEK1 mutant (MEK-AA), which inhibits ERK activity, completely abolished RTA induction by 15026 (FIG. 9D). These data demonstrate that the ERK pathway is required for KSHV reactivation downstream of microtubule perturbation.

### Example 7

### UCLB-15026 Activates LTR Transcriptional Activity Via the ERK Pathway In Vitro and Ex Vivo, and Potentiates Inducing Effect of Prostratin and Valproic Acid

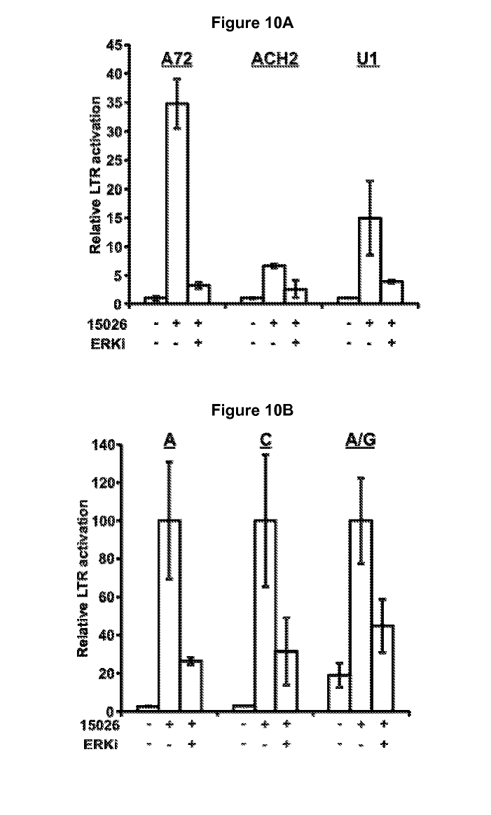

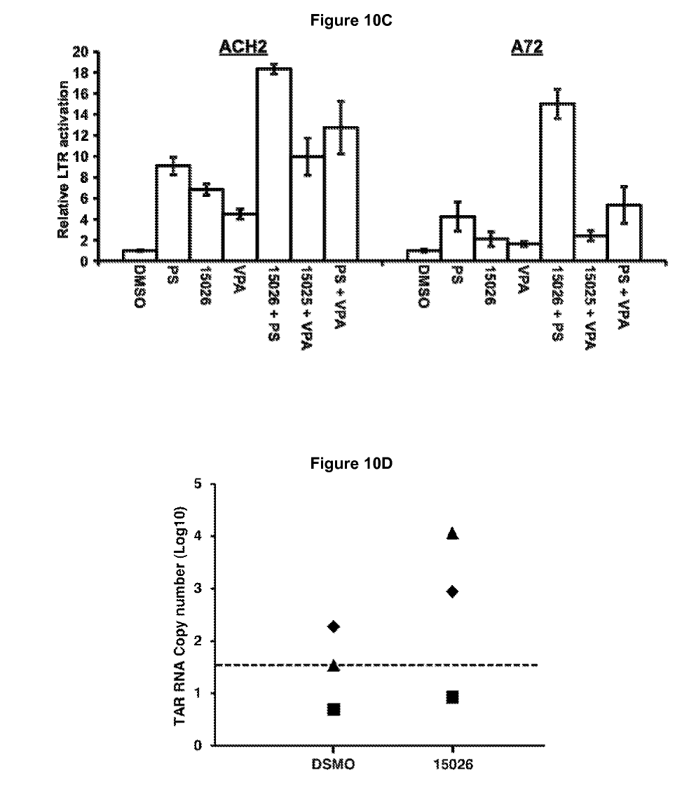

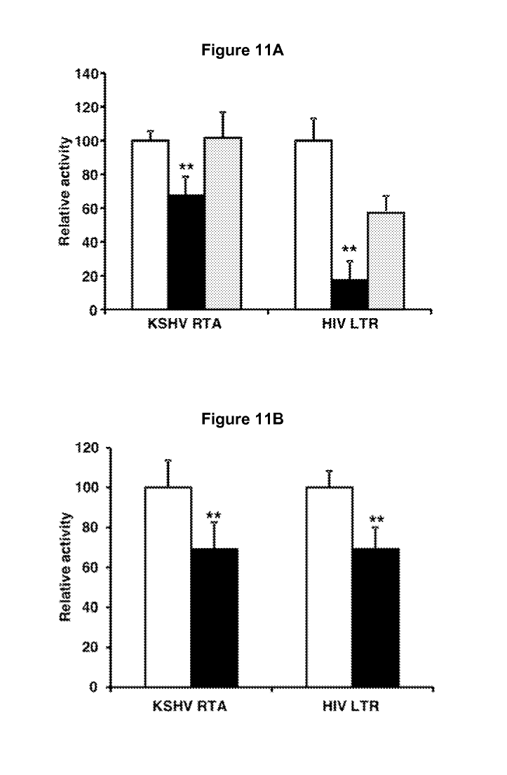

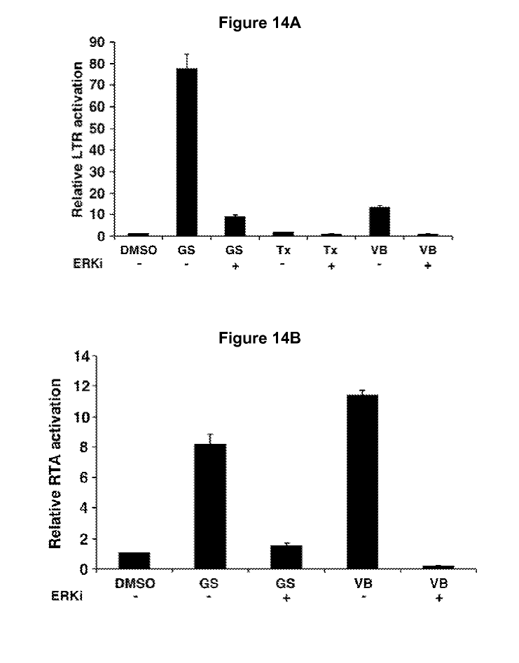

Some small molecule activators can disrupt latency of different virus families, notable examples include protein kinase C activators such as prostratin and histone deacetylase inhibitors such as valproic acid (VPA)⁴⁷⁻⁵⁰. We therefore examined whether 15026 can also activate HIV-1 using the HIV latency models J-LAT, ACH2 and U1 cell lines⁵¹⁻⁵⁴, and whether UCLB-15026 acts through the ERK pathway as for KSHV. The J-Lat cell line A72 is a cell clone derived from Jurkat T cells harboring a transcriptionally silent HIV-1 LTR-GFP construct. Cell lines ACH2 and U1 are subclones derived from T-cell and promonocyte, respectively, with both lines infected by full-length HIV-1 but with minimal virus production unless they are activated by cytokines or chemicals^{53,54}. Treatment of A72, ACH2 or U1 cells with 15026 induced LTR activity as measured by GFP (A72) or TAR RNA (ACH2 and U1) expression, and treatment of all cells with ERK inhibitor U0126 (ERKi) inhibited 15026 activity (FIG. 10A). Next we examined if 15026 can activate LTR promoter other than that of HIV-1 subtype B, as demonstrated in both ACH2 and U1 cells. Treatment of clonal cell lines, each containing different variants of the LGIT virus encoding GFP driven by the LTR of either subtype A, C or A/G, with UCLB-15026 induced GFP expression in an ERK-dependent manner (FIG. 10B). We also showed other antimicrotubule agents including genistein and vinblastine can induce both HIV-LTR (FIG. 14A) and KSHV RTA (FIG. 14B) in an ERK dependent manner. Considering 15026 targets the ERK pathway which is distinct to protein kinase C and histones, we examined if 15026 can act synergistically with prostratin and VPA. Treatment of ACH2 cells with prostratin, 15206, or VPA all induced LTR activity (FIG. 10C). Importantly, 15026 can potentiate the effect when combined with either prostratin or VPA. The combination of 15026 with prostratin achieved higher levels of LTR induction than prostratin plus VPA. Similar results were observed in the J-LAT A72 model (FIG. 10C). We next evaluate if 15026 can induce HIV transcription in an ex vivo model. Three HIV-positive individuals were selected for this study based on defined criteria for subjects with “latent” HIV infection⁵⁵, namely, all volunteers were treated with ART for at least one year, had an undetectable plasma HIV-1 RNA level (below 50 copies per ml) for at least one year and had a level of CD4+T lymphocytes higher than 400 cells/mm³ of blood. We detected higher TAR RNA copies in two out of three PBMC cultures treated with 15026 compared with that of DMSO (FIG. 10D; volunteer 1, about 5-fold; volunteer 2, about 200-fold). Taken together, these data demonstrate that 15026, through the ERK pathway, increases the transcriptional activity of both the KSHV and HIV-1 promoters that control productive virus replication. For HIV-1, the activation of ERK pathway by 15026 can potentiate the inducing effects of small molecule activators that target either protein kinase C or histone modification. UCLB-15206 is also effective in activating HIV LTR ex vivo.

### Example 8

### Full Induction of KSHV RTA and HIV-1 LTR by UCLB-15026 Requires MSK1 Activity

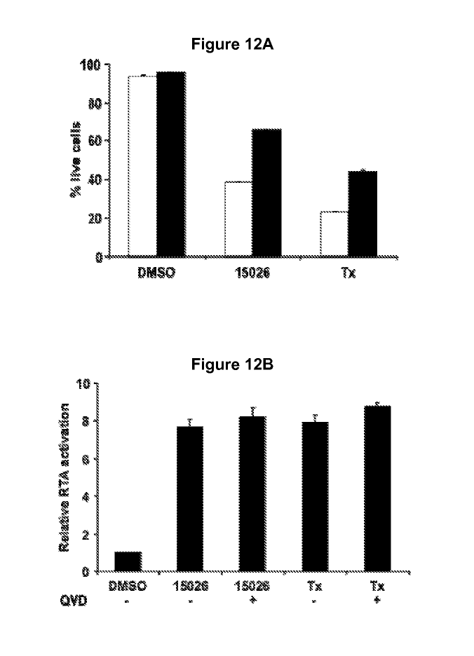

Mitogen- and stress-activated protein kinase 1 (MSK1), a known substrate of activated ERK, is a histone kinase that phosphorylates histone H3 leading to activation of polycomb-silenced gene^{56,57}. Histone modification by Polycomb repressive complex 2 is recognised to be important for maintaining KSHV latency^{58,59} and led us to examine the role of MSK1 in KSHV reactivation. We first examined the effect of chemically inhibiting MSK1 by H89 on 15026 activity. Induction of KSHV RTA and HIV LTR by 15026 was reduced by 35% and 80% compared to DMSO control, respectively (FIG. 11A). As H89 can also inhibit protein kinase A (PKA), we determined whether the use of a PKA inhibitor Rp-cAMP alone would contribute to the modulation of 15026 activity. We showed Rp-cAMP has no apparent effect on KSHV RTA induction by 15026 (FIG. 11A), whereas HIV LTR induction was reduced by about 40% (FIG. 11A) suggesting that PKA may contribute to 15026 activity in T cells. We next examined the effect of inhibiting MSK-1 on 15026 activity by ectopic expression of a dominant negative MSK-1 mutant (MSK1-DN). Induction of both KSHV RTA and HIV LTR by 15026 was significantly reduced (about 35%) in cells expressing MSK1-DN compared with the WT control (FIG. 11B). Taken together these data indicate that 15026's ability to induce KSHV RTA or HIV LTR is in part mediated through MSK1.

### Methods and Materials for Examples 6 to 8

Cell Culture.

All cell lines were grown in complete media consisting of RPMI medium 1640 (Invitrogen), with 10% FBS (BioSera) and 100 units/ml penicillin/streptomycin (Invitrogen) at 37° C. in 5% carbon dioxide, except for HEK 293T cells, which were cultured in Dulbecco's modified Eagle's medium (Invitrogen) with 10% FBS and 100 units/ml penicillin-streptomycin. Jurkat-based LGIT cell lines for examining latency reactivation for various HIV subtypes⁶⁰ were kind gifts from David Schaffer (University of California, Berkeley). BCBL-1, J-LAT A72 and U1 cells were obtained through the NIH AIDS Research and Reference Reagent Program, Division of AIDS, NIAID, NIH.

Chemicals.

Other chemicals used include vinblastine, valproic acid and Rp-cAMP (Sigma), JNK Inhibitor SP600125, p38 inhibitor SB203580, MEK/ERK inhibitors U0126 and PD0325901 (Merck), Prostratin (Cambridge Biosciences), and H89 (VWR).

### Study Subjects

Three HIV-1-positive individuals at the Mortimer Market Centre (London, UK) were selected to take part in this study on the basis of the following criteria: all volunteers were treated with ART for at least one year, had an undetectable plasma HIV-1 RNA level (below 50 copies per ml) for at least one year and had a level of CD4+ T lymphocytes higher than 400 cells/mm3 of blood. The study was approved by the Research Ethics Committee and all patients gave written informed consent.

KSHV Lytic Cycle Induction Assay.

To study the effect of MAPK-pathway specific inhibitors on KSHV RTA induction, the inhibitors were added to JSC-1 PYFP cells at least 4 hours prior to the addition of test compounds. To study the effect of constitutively active MEK (MEK-ED) or dominant negative MEK (MEK-AA) on KSHV RTA induction, JSC-1 PYFP cells were transduced with lentiviruses expressing either MEK-ED or MEK-AA, at an MOI of 10, 48 hours prior to the addition of test compounds. Levels of YFP expression were measured by flow cytometry at 40 hours post compound-treatment.

Lentiviral Vector Construction.

For lentivectors expressing MEK1 variants: ORF encoding either a constitutively active MAPK kinase 1 (MEK1) mutant (MEK-ED), or a dominant negative MEK1 mutant (MEK-AA) was sub-cloned into the pSIN-Dual-Ubi-mCherry lentivector genome (all three constructs were kind gifts from David Escors, UCL). For lentivectors expressing MSK1 variants, ORF encoding either WT MSK1, or dominant negative MSK1 (both constructs were kind gifts from Simon Arthur, University of Dundee) was subcloned into the pSIN-Dual-Ubi-mCherry lentivector genome. Lentiviral vectors expressing the MEK and MSK1 variants were produced as described previously⁶¹. Briefly, HEK 293T cells were transfected with p8.91, pMDG (both constructs were kind gifts from Didier Trono, EPFL), and lentiviral vector genome encoding MEK mutants, using FuGENE-6 (Roche). Filtered supernatants were collected at 48 and 72 hours post-transfection and titrated on JSC-1 cells.

HIV-1 LTR Induction Assays.

LTR induction was measured by monitoring the induction of either GFP expression by flow cytometry, or quantitative RT-PCR for HIV-1 TAR or GFP transcripts (both downstream of LTR promoter) and data normalised based on GAPDH levels. For GFP expression, GFP-expressing J-LAT A72 cells were measured by flow cytometry following incubation with individual compounds with or without U0126 (10 μM) for 24 hours. For examining the effect of compounds on LTR of different HIV-1 subtypes, clonal cells derived from off populations of Jurkat cells infected with different variants of the LGIT virus⁶⁰, kind gifts from David Schaffer (University of California, Berkeley), were measured by flow cytometry following incubation with individual compounds with or without U0126 (10 μM) for 24 hours. For transcript quantification, total RNA was purified and reverse transcribed as described above, and transcripts of TAR or GFP were quantified by Q-PCR and the relative fold induction was normalised based on GAPDH expression. Each reaction containing 1× Maxima™ Probe/ROX qPCR Master Mix and the relevant primers and probe combination: TAR primers were TAR forward primer, 5′-GCTAACTAGGGAACCCACTGCTT-3′ (SEQ ID NO. 5); TAR reverse primer, 5′-CAACAGACGGGCACACACTACT-3′ (SEQ. ID NO. 6); and TAR probe, 5′-[FAM]AGCCTCAATAAAGCTTGCCTTGAGTGCTTC[TAM]-3′ (SEQ ID NO. 7). GFP primers were GFP forward primer, 5′-CAACAGCCACAACGTCTATATCAT-3′ (SEQ ID NO. 8); GFP reverse primer, 5′-ATGTTGTGGCGGATCTTGAAG-3′ (SEQ ID NO. 9); and GFP probe, 5′-[FAM]CCGACAAGCAGAAGAACGGCATCAA[TAMRA]-3′ (SEQ ID NO. 10). GAPDH primers were GAPDH forward primer, 5′-GACTCATGACCACAGTCCATGC-3′ (SEQ ID NO. 11); GAPDH reverse primer, 5′-AGAGGCAGGGATGATGTTCTG-3′ (SEQ ID NO. 12); and GAPDH probe, 5′-[JOE]CATCACTGCCACCCAGAAGACTGTG[TAM]-3′ (SEQ ID NO. 13). Absolute copy number was determined with reference to a standard curve derived by Q-PCR against serial dilutions of plasmid encoding the amplicon of either TAR, GFP or GAPDH.

Ex Vivo HIV-1 LTR Induction Assays.

LTR induction was measured by monitoring the induction by quantitative RT-PCR for HIV-1 TAR and data normalised based on GAPDH levels. Non-adherent PBMCs was purified from whole blood and incubated with either DMSO or UCLB-15026 (100 μM) for 24 h. Total RNA was purified, reverse transcribed, and TAR copies were quantified as described above.

Trypan Blue Assay.

JSC-1 PYFP cells were treated with DMSO or U0126 (10 μM) for 40 h and cell viability was measured by counting cells excluding trypan blue stain.

References mentioned herein or otherwise useful for background:
42 Bhalla, K. N. Microtubule-targeted anticancer agents and apoptosis. Oncogene 22, 9075-9086 (2003).
43 Fan, M. & Chambers, T. C. Role of mitogen-activated protein kinases in the response of tumor cells to chemotherapy. Drug Resist Updat 4, 253-267 (2001).
44 Yu, F. et al. Systematic identification of cellular signals reactivating Kaposi sarcoma-associated herpesvirus. PLoS Pathog 3, e44 (2007).
45 Xie, J., Ajibade, A. O., Ye, F., Kuhne, K. & Gao, S. J. Reactivation of Kaposi's sarcoma-associated herpesvirus from latency requires MEK/ERK, JNK and p38 multiple mitogen-activated protein kinase pathways. Virology 371, 139-154 (2008).
46 Escors, D. et al. Targeting dendritic cell signaling to regulate the response to immunization. Blood 111, 3050-3061, doi:blood-2007-11-122408 [pii] 10.1182/blood-2007-11-122408 (2008).
47 Lehrman, G. et al. Depletion of latent HIV-1 infection in vivo: a proof-of-concept study. Lancet 366, 549-555 (2005).
48 Ylisastigui, L., Archin, N. M., Lehrman, G., Bosch, R. J. & Margolis, D. M. Coaxing HIV-1 from resting CD4 T cells: histone deacetylase inhibition allows latent viral expression. Aids 18, 1101-1108 (2004).
49 Kulkosky, J. et al. Prostratin: activation of latent HIV-1 expression suggests a potential inductive adjuvant therapy for HAART. Blood 98, 3006-3015 (2001).
50 Brown, H. J., McBride, W. H., Zack, J. A. & Sun, R. Prostratin and bortezomib are novel inducers of latent Kaposi's sarcoma-associated herpesvirus. Antivir Ther 10, 745-751 (2005).
51 Jordan, A., Defechereux, P. & Verdin, E. The site of HIV-1 integration in the human genome determines basal transcriptional activity and response to Tat transactivation. Embo J 20, 1726-1738 (2001).
51 Jordan, A., Bisgrove, D. & Verdin, E. HIV reproducibly establishes a latent infection after acute infection of T cells in vitro. Embo J 22, 1868-1877 (2003).
53 Clouse, K. A. et al. Monokine regulation of human immunodeficiency virus-1 expression in a chronically infected human T cell clone. J Immunol 142, 431-438 (1989).
54 Folks, T. M. et al. Characterization of a promonocyte clone chronically infected with HIV and inducible by 13-phorbol-12-myristate acetate. J Immunol 140, 1117-1122 (1988).
55 Finzi, D. et al. Identification of a reservoir for HIV-1 in patients on highly active antiretroviral therapy. Science 278, 1295-1300 (1997).
56 Lau, P. N. & Cheung, P. Histone code pathway involving H3 S28 phosphorylation and K27 acetylation activates transcription and antagonizes polycomb silencing. Proc Natl Acad Sci USA 108, 2801-2806, doi:1012798108 [pii] 10.1073/pnas.1012798108 (2011).
57 Gehani, S. S. et al. Polycomb group protein displacement and gene activation through MSK-dependent H3K27me3S28 phosphorylation. Mol Cell 39, 886-900, doi:S1097-2765(10)00630-1 [pii] 10.1016/j.molcel.2010.08.020 (2010).
58 Toth, Z. et al. Epigenetic analysis of KSHV latent and lytic genomes. PLoS Pathog 6, e1001013, doi:10.1371/journal.ppat.1001013 (2010).
59 Gunther, T. & Grundhoff, A. The epigenetic landscape of latent Kaposi sarcoma-associated herpesvirus genomes. PLoS Pathog 6, e1000935, doi:10.1371/journal.ppat.1000935 (2010).
60 Burnett, J. C. et al. Combinatorial latency reactivation for HIV-1 subtypes and variants. J Virol 84, 5958-5974, doi:JVI.00161-10 [pii] 10.1128/JVI.00161-10 (2010).
61 Besnier, C., Takeuchi, Y. & Towers, G. Restriction of lentivirus in monkeys. Proc Natl Acad Sci USA 99, 11920-11925, doi:10.1073/pnas.172384599 172384599 [pii] (2002).

All individual references mentioned herein are incorporated herein by reference in their entirety.

1-34. (canceled)

35. A method of treating a subject having a latent virus comprising administering an anti-microtubule agent and an anti-viral agent to the subject.

36. A method of treating a subject having a latent virus according to claim 35, wherein the anti-microtubule agent is a compound of the formula (I), or a pharmaceutically acceptable salt thereof or a prodrug thereof, 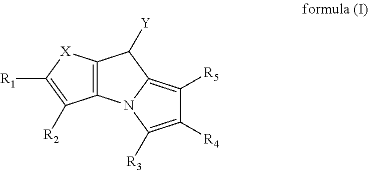
wherein X is selected from S, O, N—R₆, wherein R₆ is selected from H or optionally substituted alkyl,
—Y is selected from ═O, —OH, ═N—R₇, wherein R₇ is selected from H or optionally substituted alkyl,
R₂ is optionally substituted aryl or heteroaryl,
R₁, R₃, R₄ and R₅ are each independently selected from hydrogen, halogen, optionally substituted alkyl, optionally substituted alkoxy, optionally substituted amino group, a nitro group, optionally substituted aryl and optionally substituted heteroaryl.

37. The method of treating a subject having a latent virus according to claim 36, wherein X is S, —Y is ═O, and R₁, R₃, R₄ and R₅ are all H.

38. The method of treating a subject having a latent virus according to claim 36, wherein R₂ is a group of the formula (II) 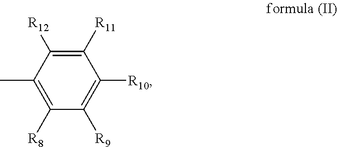
wherein R₈, R₉, R₁₀, R₁₁, R₁₂ are each independently selected from hydrogen, halogen, optionally substituted alkyl, nitro, cyano, hydroxy, optionally substituted alkoxy, optionally substituted amino, carboxy, alkoxycarbonyl, methylenedioxy, ethylenedioxy, optionally substituted alkylcarbonyloxy, optionally substituted arylalkoxy, optionally substituted acyl, optionally substituted aminocarbonyl and carboxy.

39. The method of treating a subject having a latent virus according to claim 38, wherein R₈, R₁₁, R₁₂ are each H and R₉ and R₁₀ are each independently selected from hydrogen, hydroxy and optionally substituted alkoxy, with at least one of R₉ and R₁₀ being hydroxy or optionally substituted alkoxy.

40. The method of treating a subject having a latent virus according to claim 38, wherein X is S, —Y is ═O, R₁, R₃, R₄ and R₅ are all H, R₈, R₉, R₁₁, R₁₂ are each H and R₁₀ is methoxy.

41. The method of treating a subject having a latent virus according to claim 35, wherein the anti-microtubule agent is selected from genistein, vincristine, vinblastine, vindesine, vinorelbin, taxotere, maytansin, rhizoxin, taxane compounds, and combinations thereof.

42. The method of treating a subject having a latent virus according to claim 35, wherein the anti-viral agent is an agent for treating a herpes virus.

43. The method of treating a subject having a latent virus according to claim 35, wherein the anti-viral agent is selected from cidofovir, acyclovir, valaciclovir, ganciclovir, valganciclovir, penciclovir, famciclovir, vidarabine, cytarabine, idoxuridine, trifluridine, edoxudine, brivudine, foscarnet, docosanol, fomivirsen and tromantadine, maribavir, and AIC246.

44. The method of treating a subject having a latent virus according to claim 35, wherein the anti-viral agent is an agent for treating HIV.

45. The method of treating a subject having a latent virus according to claim 35, wherein the anti-viral agent is selected from nucleoside reverse transcriptase inhibitors, non-nucleoside reverse transcriptase inhibitors, nucleotide reverse transcriptase inhibitors, protease inhibitors, maturation inhibitors, attachment inhibitors, fusion inhibitors, entry inhibitors, integrase inhibitors, zinc finger inhibitors, chemokine receptor blockers and antisense molecules, and combinations thereof.

46. The method of treating a subject having a latent virus according to claim 35, wherein the latent virus is selected from a retrovirus and a herpes virus.

47. The method of treating a subject having a latent virus according to claim 46, wherein the retrovirus is selected from HIV-1, (HIV-2), and Human T-lymphotropic virus Type I and II (HTLV-1, -2).

48. The method of treating a subject having a latent virus according to claim 46, wherein the herpes virus is selected from Epstein-Barr virus, Kaposi's sarcoma-associated herpes virus, herpes simplex virus-1, herpes simplex virus-2, varicella zoster virus, cytomegalovirus, roseolovirus, and an animal herpesvirus.

49. A composition comprising an anti-microtubule agent and an anti-viral agent.

50. A composition according to claim 49, wherein the anti-microtubule agent is a compound of the formula (I), or a pharmaceutically acceptable salt thereof or a prodrug thereof, 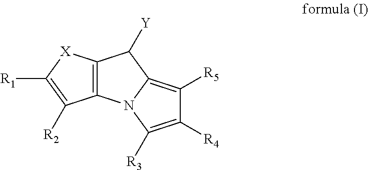
wherein X is selected from S, O, N—R₆, wherein R₆ is selected from H or optionally substituted alkyl,
—Y is selected from ═O, —OH, ═N—R₇, wherein R₇ is selected from H or optionally substituted alkyl,
R₂ is optionally substituted aryl or heteroaryl,
R₁, R₃, R₄ and R₅ are each independently selected from hydrogen, halogen, optionally substituted alkyl, optionally substituted alkoxy, optionally substituted amino group, a nitro group, optionally substituted aryl and optionally substituted heteroaryl.

51. A method of activating a latent virus in a subject comprising administering an anti-microtubule agent to the subject.

52. A method of activating a latent virus in a subject according to claim 51, wherein the anti-microtubule agent is a compound of the formula (I), or a pharmaceutically acceptable salt thereof or a prodrug thereof, 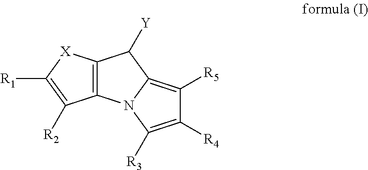
wherein X is selected from S, O, N—R₆, wherein R₆ is selected from H or optionally substituted alkyl,
—Y is selected from ═O, —OH, ═N—R₇, wherein R₇ is selected from H or optionally substituted alkyl,
R₂ is optionally substituted aryl or heteroaryl,
R₁, R₃, R₄ and R₅ are each independently selected from hydrogen, halogen, optionally substituted alkyl, optionally substituted alkoxy, optionally substituted amino group, a nitro group, optionally substituted aryl and optionally substituted heteroaryl.

53. A method of activating a latent virus in a subject according to claim 51, wherein the anti-microtubule agent is selected from genistein, vincristine, vinblastine, vindesine, vinorelbin, taxotere, maytansin, rhizoxin, taxane compounds, and combinations thereof.

54. A method of activating a latent virus in a subject according to claim 51, wherein the latent virus is selected from a retrovirus and a herpes virus.
